(19) United States
(12) Patent Application Publication
(10) Pub. No.: US 2026/0054246 A1
(43) Pub. Date: Feb. 26, 2026

(54) CATALYTIC WALL REACTOR AND METHODS OF NON-OXIDATIVE DIRECT METHANE CONVERSION TO ETHYLENE

(71) Applicants: Dongxia LIU, Newark, DE (US); Sichao CHENG, Newark, DE (US)

(72) Inventors: Dongxia LIU, Newark, DE (US); Sichao CHENG, Newark, DE (US)

(73) Assignee: University of Delaware, Newark, DE (US)

(21) Appl. No.: 19/106,460

(22) PCT Filed: Aug. 25, 2023
(86) PCT No.: PCT/US2023/031189

§ 371 Date: Feb. 25, 2025

Related U.S. Application Data
(60) Provisional application No. 63/373,550, filed on Aug. 26, 2022.

(51) Int. Cl. B01J 19/24; B01D 53/22; B01J 19/00; B01J 21/12; B01J 23/42; B01J 23/745; B01J 35/00; B01J 35/45; C01B 3/26; C01B 3/50; C07C 2/80

(52) U.S. Cl. CPC B01J 19/2415; B01D 53/22; B01J 19/0013; B01J 21/12; B01J 23/42; B01J 23/745; B01J 35/19; B01J 35/45; C01B 3/26; C01B 3/503; C07C 2/80; B01D 2256/24; B01D 2257/108; B01J 2219/00054; B01J 2219/00135; B01J 2219/00157; B01J 2219/00164; C01B 2203/02; C01B 2203/041; C01B 2203/0811; C01B 2203/085; C01B 2203/1047; C01B 2203/1082

(57) ABSTRACT

A reactor, system, and method of converting methane non-oxidatively. A thermal catalytic reactor has a non-oxidative methane coupling (NMC) catalyst disposed on a first surface of a substrate. The NMC catalyst endothermically converts methane in a reaction zone on the catalyst side of the reactor to a product mixture. The reaction zone is heated by thermal conduction. The spatial temperature profile has a sharp increase and decrease that leads to selective control of the surface methane activation and gas phase reaction propagation. The reactor also has an inlet for introducing methane gas for contacting the NMC catalyst and an outlet for removing the product mixture. The heat source may generate the process heat chemically or electrically. Temperature profiles are controlled by zoning the combustion catalyst location or conductive heating element in the reactor.

### CROSS-REFERENCE TO RELATED APPLICATION

This application claims priority to U.S. Provisional Patent Application No. 63/373,550, filed Aug. 26, 2022, the entire disclosure of which is incorporated herein by reference for all purposes.

### STATEMENT REGARDING FEDERALLY SPONSORED RESEARCH

This invention was made with government support under Grant No. CBET-5247380 awarded by the National Science Foundation and Grant No. DE-FE0031877 awarded by the Department of Energy, Office of Fossil Energy. The government has certain rights in the invention.

### BACKGROUND OF THE INVENTION

Natural gas abundance, boosted with the shale gas revolution, offers substantial economic opportunities. However, low temperatures and high pressures are required to collect and transport methane (CH₄), the primary component of natural gas, to centralized facilities for further processing. Instead, modular upcycling offers a path for off-site processing of CH₄ into higher value hydrocarbon products. Unlike state-of-the-art centralized gas-to-liquid (GTL) plants-—where multi-step and multi-unit operations such as the indirect reforming to syngas followed by Fischer-Tropsch synthesis of higher hydrocarbons are utilized—modular upcycling requires single-step pathways for direct conversion of CH₄ into higher hydrocarbons. In this regard, non-oxidative methane coupling (NMC) is a viable pathway as it enables single step olefin and aromatic production generating hydrogen (H₂) as a co-product.

Traditionally, the NMC reaction has faced challenges, including low CH₄ conversion, low product yield, and catalyst durability, due to the endothermic nature of the reaction, coke formation and catalyst deactivation. Current research efforts aim to enhance the mechanism understanding of the process, optimize catalyst formulations, and develop efficient reactor systems to pave the way for a more sustainable and economically viable methane conversion technology. The state-of-the-art for non-oxidative methane conversion (NMC) has seen significant advancements in recent years, focusing on the development of novel catalysts and reactor designs to overcome the challenges associated with this highly endothermic process. Metal-loaded zeolite catalysts (such as molybdenum/ZSM-5 zeolite (Mo/ZSM-5)) and iron-modified silica (Fe/SiO₂) catalysts have demonstrated promising performance in NMC, with the latter exhibiting high CH₄ conversion and C2+ yields at elevated temperatures. However, issues such as low conversion rates, rapid catalyst deactivation, and the necessity for high heat supply remain challenges for the widespread adoption of NMC. A mechanistic investigation into the Fe/SiO₂ catalyst revealed that a complex heterogeneous-homogeneous reaction network underlies its performance. The silica lattice-confined Fe species initiate CH₄ dehydrogenation to generate methyl and hydrogen species, enabling a series of subsequent surface and gas-phase reactions to form dehydrogenated and cyclized larger hydrocarbon products. Based on this mechanistic understanding, thermal engineering could be a potential strategy for tailoring NMC reaction performance towards lighter hydrocarbons by quenching the subsequent formation of larger hydrocarbons. However, typical upcycling processes via non-catalytic pyrolysis or catalytic fixed-beds rely on heating sources to create a uniform temperature distribution in the heating zone, where opportunities to engineer temperature profiles are minimal. As a proof of concept, independent furnaces heating upstream and downstream of a catalytic fixed bed have shown to influence NMC performance. However, as a major focus has been to develop new catalysts or gain a mechanistic understanding, where fixed beds and membrane reactors are the configurations of choice reactor designs leveraging these mechanistic insights remain unexplored. The development of more efficient and economically viable chemical reactor systems for NMC is crucial for the successful implementation of this technology. Hence, there is a need for improving NMC performance by engineering the thermal driving force in a new reactor design.

### SUMMARY OF THE INVENTION

Disclosed herein is a millisecond catalytic wall reactor that resolves these challenges with thermal self-sustainability. The engineered reactor, coated with NMC and combustion catalysts on opposite tube wall surfaces, enables milli-second activation of methane powered by the energy generated from catalytic combustion of fuel gas with greatly improved heat transfer by removing the thermal boundary layers and controlled reaction zone with sharp temperature gradient, which inhibits the heavy product or coke formation. This reactor innovation leads to unprecedented NMC performance with high single-pass CH₄ conversion, >99% C2+ selectivity, negligible coke formation, and autothermal operation.

In an aspect of the invention, a reactor comprises (i) a thermal catalytic reactor member comprising a non-oxidative methane coupling (NMC) catalyst disposed on a first surface of a substrate, wherein the NMC catalyst is configured to endothermically convert methane in a reaction zone on the NMC catalyst side of the thermal catalytic reactor member to a product mixture comprising hydrogen and a C₂₊ hydrocarbon, and (ii) a source of process heat configured to deliver heat to the reaction zone by thermal conduction through the thermal catalytic reactor member. The reactor also comprises a first inlet for contacting the NMC catalyst with methane gas, and a first outlet for removal of product mixture from the reactor.

In an embodiment of the reactor, the source of process heat comprises one or more of (i) a combustion catalyst configured to generate the process heat by an exothermic combustion reaction in the presence of a combustion fuel; and a conductive heating element configured to generate the process heat by Joule heating upon passage of electrical current through the conductive heating element.

In another embodiment of the reactor, the thermal catalytic reactor member comprises one or more substrates defining the first surface and a second surface disposed on opposite sides of the one or more substrates and isolated from the NMC catalyst layer, and wherein the combustion catalyst is disposed on the second surface of the one or more substrates.

In an embodiment, the reactor includes a reactor housing having a tubular configuration defined by the one or more substrates forming one or more walls for containing the methane gas, the first surface comprises an inner surface of the one or more walls, and the second surface comprises the outer surface of the one or more walls.

In another embodiment, the reactor includes a reactor housing comprising a tubular reactor defined by one or more inner channels for contacting the combustion fuel with the combustion catalyst disposed on the inner surface of the tubular reactor housing and the NMC catalyst is disposed on the outer surface of the tubular reactor.

In some embodiments, the combustion catalyst comprises a transition metal and/or a metal oxide, on a support. The transition metal catalyst comprises platinum (Pt), palladium (Pd), rhodium (Rh), ruthenium (Ru), nickel (Ni), copper (Cu), iron (Fe), cobalt (Co), silver (Ag), iridium (Ir), gold (Au) or their alloys. In other embodiments, the metal oxide comprises cerium oxide (CeO₂), zirconium oxide (ZrO₂), manganese Oxide (MnOₓ), vanadium oxide (V₂O₅), copper oxide (CuO), chromium oxide (Cr₂O₃), Cobalt oxide (CO₂O₄), iron oxide (Fe₂O₃) or mixtures thereof. The support for the transition metal and/or the metal oxide may comprise alumina, silica, magnesium oxide, barium oxide, strontium oxide, lanthanum oxide, scandium oxide, titanium oxide, vanadium oxide, chromium oxide, manganese oxide, zirconium oxide, niobium oxide (Nb₂O₅), zinc oxide, bismuth oxide or mixtures thereof.

Suitable combustion fuel may comprise methane, hydrogen, biogas, natural gas, fossil oil, biomass, or mixtures thereof.

In another aspect of the reactor, the source of process heat comprises the conductive heating element, and wherein (a) the first surface of the thermal catalytic reactor member on which the NMC catalyst is disposed comprises a surface of the conductive heating element, or (b) the conductive heating element comprises a first member of the thermal catalytic reactor member in thermal communication with a second member of the thermal catalytic reactor member that defines the first surface on which the NMC catalyst is disposed.

In an embodiment of the reactor, the conductive heating element is in a geometry of one or more hollow fibers, wires, rods, strips, plates, tubes, meshes, or monoliths.

In another embodiment, the reactor includes a reactor housing having a tubular configuration defined by one or more sidewalls for containing the methane gas, and wherein the conductive heating element is disposed inside the reactor housing and is isolated from the one or more sidewalls. In some embodiments, the catalytic reactor is tubular reactor having a cylindrical geometry defining a longitudinal axis and the conductive heating element is disposed along the longitudinal axis.

In various embodiments, the C2+ hydrocarbons comprise one or more of acetylene, ethylene, ethane, benzene, toluene, naphthalene, and coke.

In some embodiments, the conductive heating element comprises a conductive ceramic, a metal carbide, a metal nitride, a two-dimensional MXene material, a metal, an alloy, a conductive carbon, or a combination thereof.

The conductive ceramic may comprise mixed metal oxides of perovskite-type oxide conductor represented by a formula of M′Ce_{1-x-y}ZrₓM″_{y}O_{3-δ}, where
M′ is Sr or Ba,
M″ is at least one of Ti, V, Cr, Mn, Fe, Co Ni, Cu, Nb, Mo, W, Pr, Nd, Pm, Sm, Eu,
Gd, Tb, Dy, Ho, Er, Tm and Yb,
x is in the range of 0.1 and 0.2, inclusive,
y is in the range of 0.1 and 0.3, inclusive,
δ is in the range of 0 to 0.7,
wherein the metal carbide comprises silicon carbide (SIC), tungsten carbide (WC), Titanium Carbide (TIC), Zirconium Carbide (ZrC), Hafnium Carbide (HfC), Niobium Carbide (NbC), titanium aluminum carbide (Ti₂AlC), and the like;
wherein the metal nitride comprises titanium nitride (TiN), tantalum nitride (TaN), vanadium nitride (VN), molybdenum nitride (MoN), Ti₂NCd, Ti₂NAl, and the like,
wherein the two-dimensional MXene material comprises Ti₂C, Nb₂C, (Ti_{2-y}Nb_{y})C, Ti₂N, Nb_{1.33}C, and the like;
wherein the metal selected from the group consisting of tungsten, molybdenum, nickel, copper, chromium, titanium, silver, tungsten, tantalum, and
wherein the alloy selected from the group consisting of nichrome (Ni₈₀Cr₂O), constantan (CuNi₄₄), manganin (CuMn₁₂Ni), nitinol (NiTi), nichrome-V (Ni₆₀Cr₁₆Si₁₄B), copper-nickel (CuNi) alloy, iron-chromium-aluminum (FeCrAl) alloy, and stainless steel.

In embodiments of the reactor as disclosed hereinabove, the NMC catalyst layer comprises single metal atoms, sub-nanometer clusters of metals on a support, metal nanoparticles on a support. In an embodiment, the metal comprises one or more of Li, Na, K, Mg, Al, Ca, Sr, Ba, Y, La, Ti, Zr, Ce, Cr, Mo, W, Re, Fe, Co, Ni, Cu, Zn, Ge, In, Sn, Pb, Bi, and Mn, and the support comprises quartz, quartz melt, cristobalite, silicate-1 vitreous silica, and zeolite.

In an embodiment, the reactor further comprises a hydrogen separation membrane disposed in proximity to the NMC catalyst layer. The hydrogen separation membrane may comprise one or more a hollow fiber membrane, a carbon molecular sieve membrane or a mixed metal oxide ceramic membrane.

In some embodiments, the hydrogen separation membranes has a pore structure tuned to exclude sorption of one or more coke precursors.

In an embodiment of the reactor as disclosed hereinabove, the thermal catalytic reactor member has a geometry selected from the group consisting of tubular, multi-tubular, plate, honeycomb.

In various embodiments, the reactor further comprises a second inlet for contacting a combustion fuel over the combustion catalyst; and a second outlet for removal of combustion product mixture from the reactor.

In an aspect, a system comprises the reactor as disclosed hereinabove; and a first gas flow controller configured to control flow-rate of methane gas through the first inlet over the NMC catalyst.

In an embodiment, the system further comprises a second gas flow controller configured to control flow rate and direction of flow of the combustion fuel over the combustion catalyst.

In another embodiment, the system further comprises a current controller configured to control the amount of electrical current through the conductive heating element.

In another aspect, a method of converting methane non-oxidatively comprises the steps of (a) providing the reactor as disclosed hereinabove; (b) contacting the NMC catalyst layer, in the reaction zone, with methane at a desired flow rate, to produce the product mixture comprising hydrogen and a C₂₊ hydrocarbon; and (c) chemically or electrically generating an amount of process heat with the source of process heat to thereby deliver the process heat to the reaction zone on the first surface, wherein the process heat creates a temperature in the range of 850° C. to about 1150° C. at the reaction zone, thereby resulting in a non-uniform temperature profile inside the reactor, where the temperature decreases at a rate in the range of 1 to 200° C./mm away from the thermal catalytic reactor member.

The C₂₊ hydrocarbons comprise acetylene, ethylene, ethane, benzene, toluene, naphthalene, or a combination thereof.
in some embodiments, the flow rate of methane is in the range of 5 ml/min to about 500 mL/min.

In an embodiment, the source of process heat comprises a combustion catalyst on the second surface, and the step of chemically generating heat on the second surface comprises contacting a gas containing oxygen with the combustion catalyst.

In yet another embodiment, the method further comprises controlling one or more of (i) the flow rate of methane and the gas, (ii) direction of flow of the gas to regulate a percentage of methane converted and/or a selectivity of the methane conversion to one or more of the products, and (iii) catalytic wall length.

In an embodiment, the source of process heat comprises an electrically conductive heating element, and the step of electrically generating the process heat comprises passing an electrical current through the conductive heating element. The method further comprises controlling an amount of electrical current or power to maintain the temperature of the reaction zone to thereby regulate a percentage of methane converted and/or a selectivity of the methane conversion to one or more of the products.

In an embodiment of the method, the percentage of methane converted is greater than 5%; an yield of C₂₊ hydrocarbons is greater than 4.5%; and the coke is produced in an amount of 10% or less.

In another embodiment, the method further comprises separating hydrogen from the product mixture by a hydrogen separation membrane disposed in proximity to the NMC catalyst layer.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIG. 1A shows a schematic illustration of an exemplary reactor comprising a thermal catalytic wall reactor member having a non-oxidative methane coupling (NMC) catalyst disposed on the inside wall and a combustion catalyst on the outside wall of the thermal catalytic wall reactor member, according to an embodiment of the present invention.

FIG. 1B displays a schematic illustration of a portion of the exemplary thermal catalytic wall reactor member shown in FIG. 1A, according to an embodiment of the present invention.

FIG. 1C shows a schematic illustration of an exemplary reactor comprising a thermal catalytic wall reactor member having an NMC catalyst disposed on the outside wall and a combustion catalyst on the inside wall of the thermal catalytic wall reactor member, according to an embodiment of the present invention.

FIG. 1D displays a schematic illustration of a portion of the exemplary thermal catalytic wall reactor member shown in FIG. 1C, according to an embodiment of the present invention.

FIG. 2A shows a schematic illustration of another exemplary reactor comprising a plurality of thermal catalytic wall reactor members, each having an NMC catalyst disposed on the inside wall and a combustion catalyst on the outside wall of the thermal catalytic wall reactor member, according to an embodiment of the present invention.

FIG. 2B displays a schematic illustration of a portion of the exemplary thermal catalytic wall reactor member shown in FIG. 2A, and a cross-sectional view of the portion of the thermal catalytic reactor member, where the source of process heat is a combustion catalyst coating, disposed on the inside wall, configured to generate the process heat by an exothermic combustion reaction and a temperature profile generated during operation.

FIG. 2C shows a schematic illustration of yet another exemplary reactor comprising a plurality of thermal catalytic wall reactor members, each having an NMC catalyst disposed on the outside wall and a combustion catalyst on the inside wall of the thermal catalytic wall reactor member, according to an embodiment of the present invention.

FIG. 2D displays a schematic illustration of a portion of the exemplary thermal catalytic wall reactor member shown in FIG. 2C, and a cross-sectional view of the portion of the thermal catalytic reactor member, where the source of process heat is a combustion catalyst coating, disposed on the inside wall, configured to generate the process heat by an exothermic combustion reaction and a temperature profile generated during operation.

FIG. 3A shows a schematic illustration, with a partial cross-sectional view, of an exemplary reactor comprising a thermal catalytic wall reactor member having an NMC catalyst disposed on the inside wall of the thermal catalytic wall reactor member and a conductive heating element as a source of process heat, according to an embodiment of the present invention.

FIG. 3B shows a cross-sectional view of a portion of the exemplary thermal catalytic wall reactor member shown in FIG. 3A, where the wall is configured as a conductive heating element, according to an embodiment of the present invention.

FIG. 3C shows a cross-sectional view of a portion of the exemplary thermal catalytic wall reactor member shown in FIG. 3A, where the conductive heating element is disposed on the outside wall of the thermal catalytic wall reactor member, according to an embodiment of the present invention.

FIG. 4A shows a schematic illustration, with a partial cross-sectional view, of an exemplary reactor comprising a thermal catalytic reactor member having (i) an NMC catalyst and (ii) a conductive heating element configured to be a source of process heat, according to an embodiment of the present invention.

FIG. 4B shows a cross-sectional view of a portion of the exemplary thermal catalytic wall reactor member shown in FIG. 4A, where the NMC catalyst is disposed on one side of a substrate of the thermal catalytic wall reactor member, according to an embodiment of the present invention.

FIG. 4C shows a cross-sectional view of a portion of the exemplary thermal catalytic wall reactor member shown in FIG. 3A, where the NMC catalyst is disposed on at least two sides of a substrate of the thermal catalytic wall reactor member, according to an embodiment of the present invention.

FIG. 5 shows a schematic illustration of an exemplary process of fabricating a thermal catalytic reactor member, where the source of process heat is a combustion catalyst coating, disposed on the outside wall of a tubular thermal reactor member and a photograph of an exemplary reactor, according to an embodiment of the present invention.

FIG. 6 shows (a) photograph of autothermal millisecond catalytic wall reactor (MCWR) with Fe/SiO₂ and Pt/Al₂O₃ coated zones for coupling NMC and fuel combustion reactions; (b) morphology of Fe/SiO₂ catalyst region in inner tube surface of autothermal MCWR; and (c) morphology of Pt/Al₂O₃ catalyst region on the exterior surface of the reactor wall.

FIG. 7 shows Scanning electron microscopy (SEM) images showing a cross-sectional view of autothermal MCWR, where Pt/Al₂O₃ catalyst layer is on the exterior surface of reactor wall and Fe/SiO₂ catalyst layer is fused onto the inner wall surface.

FIG. 8 shows reactor schematic and parameters of autothermal catalytic wall reactor used for COMSOL simulation. A co-current flow is illustrated.

FIG. 9 shows axial temperature comparison of steady-state simulation and experiment results at center line of combustion chamber with flow rates of 145.2 and 171.6 L/h (CH₄: Air ratio=0.1). The arrow indicates the co-current flow in the NMC chamber.

FIG. 10 shows (a) cross sectional view of reactor configurations (i) blank; (ii) fixed-bed; (iii) MWCR and (iv) autothermal MWCR. (b)CH₄ conversion and product selectivity in NMC in reactor configurations from (a) in sequence. (c) Measured temperature profiles in reactors heated by an electric furnace (top) and fuel combustion (bottom) controlled to the same peak temperature; and (d) NMC performance time-on-stream in autothermal MCWR. All data (a-c) was collected under steady state conditions. (NMC reaction: 0.48 L h⁻¹ gas flow with CH₄:Ar ratio=7, 101.325 kPa pressure, 0.375 g Fe/SiO₂ catalyst in fixed-bed reactor, 0.2 g Fe/SiO₂ catalyst in MCWR, Fe concentration=0.075 wt %; fuel combustion: 132.0 L h⁻¹ gas flow with CH₄: Air ratio=0.1, 101.325 kPa pressure, 0.1 g Pt/Al₂O₃ catalyst, Pt concentration=1.0 wt %)

FIGS. 11A-11F show the effects of gas flow rate and direction on methane conversion and product selectivity in NMC in the autothermal MCWR. FIG. 11A shows temperature profile as a function of axial position in the reactor and FIG. 11B shows methane conversion and product selectivity as a function of methane flow rate in the NMC channel at a fixed combustion fuel flow rate of 145.2 L h⁻¹ gas flow (CH₄: Air ratio=0.1) under co-current flow. FIG. 11C shows temperature profile as a function of axial position in the reactor and FIG. 11D shows methane conversion and product selectivity of varying methane flow rate in the NMC channel at a fixed combustion gas flow rate of 145.2 L h⁻¹ gas flow (CH₄: Air ratio=0.1) under counter-current flow. FIG. 11E shows varying combustion gas flow rate at a fixed NMC gas flow rate of 0.48 L h⁻¹ gas flow (CH₄:Ar ratio=7). FIG. 11F shows maximized NMC performance by adjusting both NMC and fuel combustion flow rates.

FIG. 12A shows modeled local temperature in the NMC chamber as a function of iron silica catalyst coating thickness. FIG. 12B shows radial temperature profile across the whole reactor at 130 mm axial position for combustion chamber gas flow of 145.2 L h⁻¹ (red; bottom line) and 171.6 L/h (blue; top line) (NMC channel:Ar flow 0.48 L h⁻¹, coating thickness 0.55 mm; combustion channel CH₄: Air ratio=0.1, 101.325 kPa pressure; co-current flow mode.)

### DETAILED DESCRIPTION OF THE INVENTION

The present invention discloses a reactor comprising a thermal catalytic reactor member, a system comprising the reactor, and a method of converting methane non-oxidatively.

As used herein, the term “C₂₊ hydrocarbon” refers to hydrocarbons having two or more carbon atoms, including, but not limited to, acetylene, ethylene, ethane, propane, butane, heptane, benzene, toluene, xylene, and naphthalene.

As used herein, the term “autothermal” operation refers to an operation of the system where the heat used to drive one reaction (e.g., a non-oxidative methane conversion (NMC)) is provided by a simultaneous or substantially simultaneous exothermic reaction. In some embodiments, the exothermic reaction comprises combustion of hydrogen and oxygen to form water.

As used herein, the term “autothermal catalytic reactor member” is used interchangeably with” autothermal millisecond catalytic wall reactor (MCWR)” or “autothermal catalytic wall reactor” and refers to a thermal catalytic reactor member having an NMC catalyst on one side of a substrate and a combustion catalyst on the other side of the substrate.

As used herein, the term “fuel” is used interchangeably with “combustion fuel” and “combustion gas” and refers to methane, hydrogen, biogas, natural gas, fossil oil, biomass, oxygen containing compound, and mixtures thereof. In various embodiments, combustion fuel comprises air or oxygen in addition to fuel such as methane, hydrogen, biogas, natural gas, fossil oil, biomass, oxygen containing compound, and mixtures thereof

As used herein, the term “oxygen-containing compound” is used interchangeably with “gas containing oxygen” and refers to any compound having at least one atom of oxygen and capable of releasing the oxygen to react with hydrogen (e.g., to form water). In some embodiments, the oxygen-containing compound can comprise oxygen, CO₂, H₂O, alcohols (e.g., methanol, ethanol, isopropanol, etc.).

As used herein the term “biogas” refers to a mixture of mainly methane and CO₂ along with other trace gases, produced by fermentation of organic matter.

As used herein the term “natural gas” refers to a naturally occurring mixture consisting primarily of methane, along with ethane, butane and propane, found in geologic formations beneath the earth's surface.

As used herein the term “fossil oil” is used interchangeably with “crude oil” and refers to a mixture of hydrocarbons that exists in liquid phase in natural underground reservoirs and remains liquid at atmospheric pressure.

### Reactor

In an aspect of the present invention, there is provided a reactor comprising a thermal catalytic reactor member comprising an NMC catalyst, a first inlet for contacting the NMC catalyst with methane gas, and a first outlet for removal of product mixture from the reactor. The thermal catalytic reactor member comprises an NMC catalyst disposed on a first surface of a substrate, and a source of process heat configured to deliver heat to the reaction zone by thermal conduction through the thermal catalytic reactor member. In an embodiment, the NMC catalyst is configured to endothermically convert methane in a reaction zone on the NMC catalyst side of the thermal catalytic reactor member to a product mixture comprising hydrogen and a C₂₊ hydrocarbon.

In an embodiment, NMC catalyst disposed on a first surface of a substrate has a layer thickness in the range of 5 nm to 5 mm, or 500 nm to 3 mm, or 1 μm to 1 mm.

In an embodiment, the source of process heat comprises a combustion catalyst coating configured to generate the process heat by an exothermic combustion reaction in the presence of a combustion fuel. In an embodiment, combustion catalyst coating has a thickness in the range of 5 nm to 5 mm, or 500 nm to 3 mm, or 1 μm to 1 mm, or 10 μm to 100 μm.

In another embodiment, the source of process heat comprises a conductive heating element configured to generate the process heat by Joule heating upon passage of electrical current through the conductive heating element. In yet another embodiment, the source of process heat comprises both the combustion catalyst and the conductive heating element.

In some embodiments, the reactor further comprises a second inlet for contacting a gas containing oxygen over the combustion catalyst, and a second outlet for removal of combustion product mixture from the reactor.

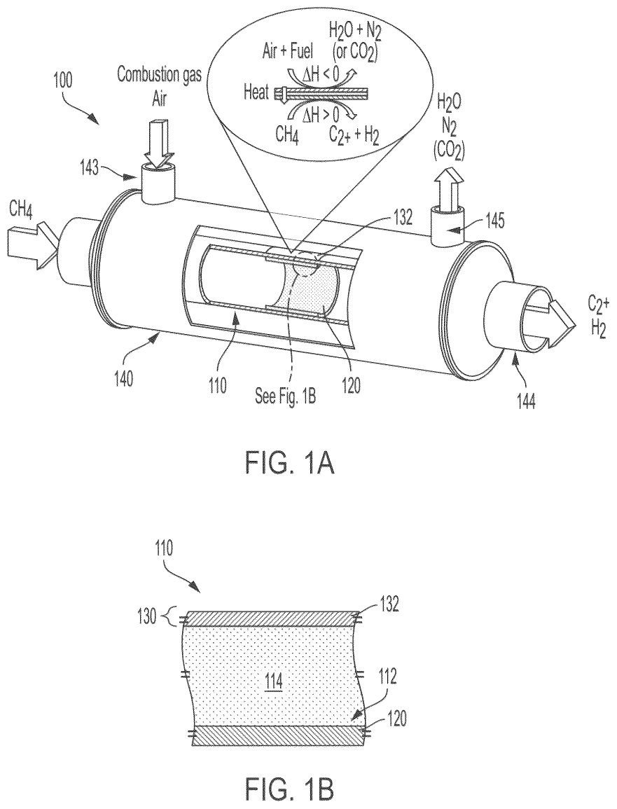

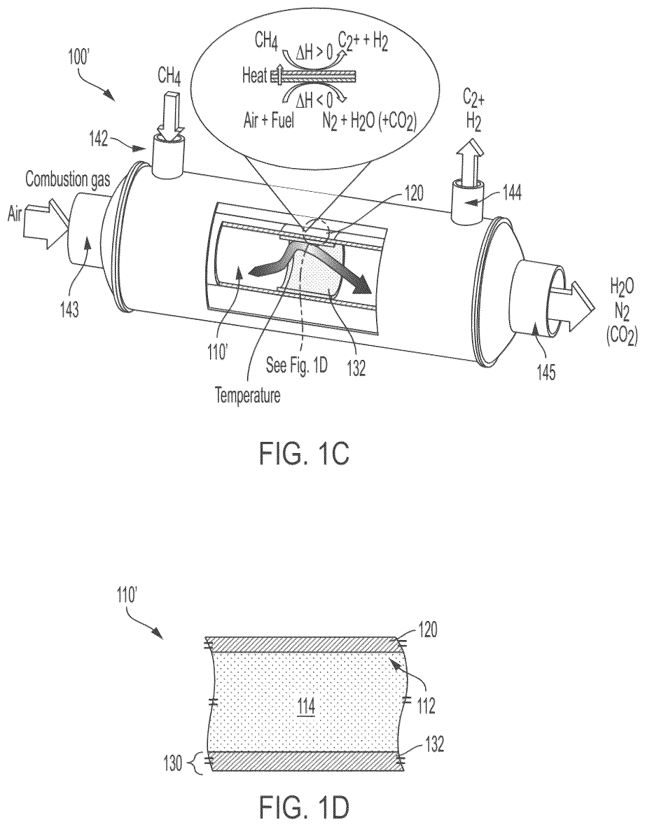

FIG. 1A shows a schematic illustration of an exemplary reactor 100 in a tubular configuration, according to an embodiment of the present invention. However, the reactor comprises a housing 140 in any suitable configuration, including, but not limited to tubular, multi-tubular, circular, rectangular, polygonal, and honeycomb. The reactor 100, as shown in FIG. 1A comprises two concentric tubes. The outer tube forms the housing 140 of the reactor 100. The housing 110 comprising the first inlet 142 and an outlet 144. The inner tube forms the thermal catalytic reactor member 110, such that an inside wall of the inner tube 110 forms the first surface comprising the NMC catalyst. The inlet 142 allows a feed stream containing methane gas to contact with the NMC catalyst and the outlet 144 allows the product, formed as a result of the non-oxidative coupling of methane in a reaction zone on the inside wall of the inner tube 110, to leave the housing 140.

FIG. 1B shows a portion of the inner tube 110 which is the exemplary thermal catalytic reactor member 110, according to an embodiment of the present invention. As shown in FIG. 1B, the NMC catalyst 120 is disposed on a first surface 112 of a substrate 114 and a combustion catalyst 132, which is the source 130 of process heat, is disposed on the outside wall of the inner tube/thermal catalytic wall reactor member 110. The NMC catalyst 120 is configured to endothermically convert methane in a reaction zone on the NMC catalyst side of the thermal catalytic reactor member to a product mixture comprising hydrogen and a C₂₊ hydrocarbon. During the reactions, the heat generated from the catalytic combustion is transferred across the quartz wall to drive the endothermic NMC reaction occurring inside the tube.

FIG. 1C shows a schematic illustration of another exemplary tubular reactor 100, which is a variation of the reactor 100 shown in FIG. 1A. In FIG. 1C, the NMC catalyst is disposed on the outside wall of the inner tube/thermal catalytic wall reactor member 110′ and the combustion catalyst is disposed on the inside wall of the inner tube/thermal catalytic wall reactor member 110′. FIG. 1D shows a schematic illustration of a portion of the exemplary innertube/thermal catalytic wall reactor member 110′ shown in FIG. 1C.

The reactors 100, 100′, as shown in FIGS. 1A and 1C further comprise a second inlet 143 for contacting a combustion fuel over the combustion catalyst, and a second outlet 145 for removal of combustion product mixture from the reactor 100, 100′.

In an embodiment of the present invention, the thermal catalytic reactor member is an autothermal catalytic reactor member comprising one or more substrates defining the first surface and a second surface disposed on opposite sides of the one or more substrates and isolated from the NMC catalyst layer. In an embodiment, the combustion catalyst is disposed on the second surface of the one or more substrates.

In another embodiment, the reactor includes a reactor housing having a tubular configuration defined by the one or more substrates forming one or more walls of the reactor housing for containing the methane gas, the first surface comprises an inner surface of the one or more walls, and the second surface comprises the outer surface of the one or more walls.

In an embodiment, the reactor includes a reactor housing comprising a tubular reactor defined by one or more inner channels for contacting the combustion fuel with the combustion catalyst disposed on the inner surface of the tubular reactor housing and the NMC catalyst is disposed on the outer surface of the tubular reactor.

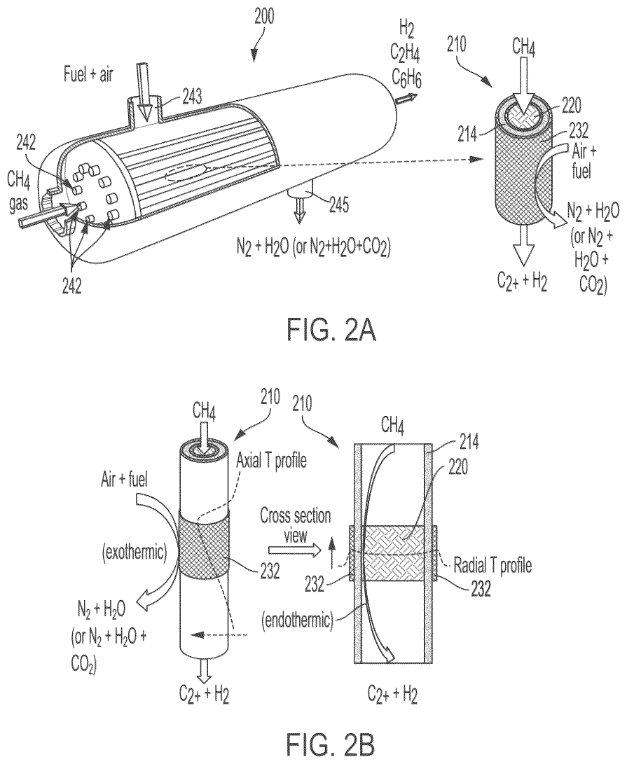

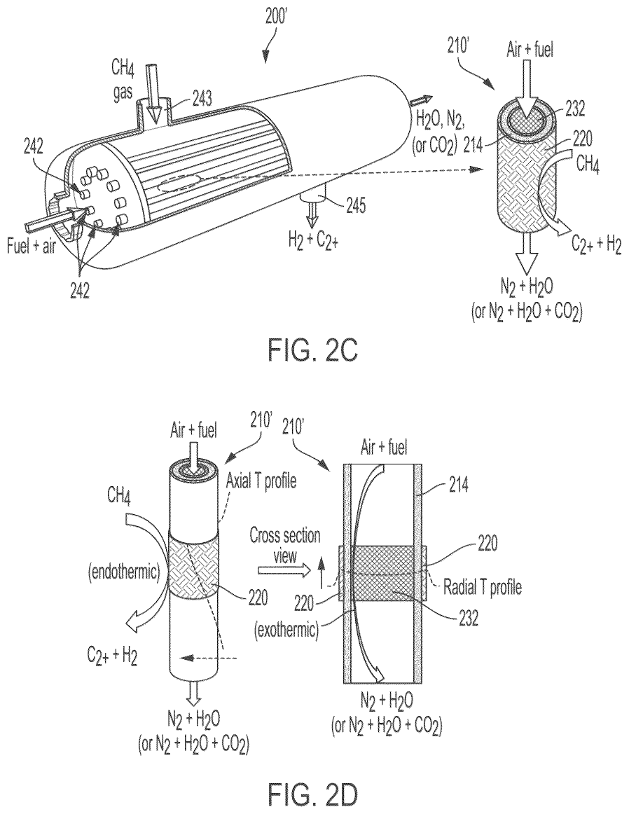

FIG. 2A shows a schematic illustration of another exemplary reactor 200 comprising a plurality of thermal catalytic wall reactor members 210. As shown in FIG. 2A, the reactor 200 is formed of an outer tube forming a housing 240 and a plurality of inner tubes forming a plurality of thermal catalytic wall reactor members 210, each of the plurality of inner tube comprises an NMC catalyst 220 disposed on the inner side of the wall 214 of the inner tube and a combustion catalyst 232 disposed on the outer side wall of the inner tube/thermal catalytic wall reactor member 210.

FIG. 2B displays a schematic illustration of a portion of the exemplary thermal catalytic wall reactor member shown in FIG. 2A, and a cross-sectional view of the portion of the thermal catalytic reactor member, where the source of process heat is a combustion catalyst coating, disposed on the inner side of the wall 214 of the inner tube/thermal catalytic wall reactor member 210, configured to generate the process heat by an exothermic combustion reaction and a temperature profile generated during operation.

FIG. 2C shows a schematic illustration of yet another exemplary reactor comprising a plurality of thermal catalytic wall reactor members 210′, each having an NMC catalyst disposed on the outer side of the wall and a combustion catalyst on the inner side of the wall of the inner tube/thermal catalytic wall reactor member 210′, according to an embodiment of the present invention.

FIG. 2D displays a schematic illustration of a portion of the exemplary thermal catalytic wall reactor member shown in FIG. 2C, and a cross-sectional view of the portion of the thermal catalytic reactor member, where the source of process heat is a combustion catalyst coating, disposed on the inside wall, configured to generate the process heat by an exothermic combustion reaction and a temperature profile generated during operation.

The reactors 200, 200′, as shown in FIGS. 2A and 2C further comprises a second inlet 243 for contacting a combustion fuel over the combustion catalyst, and a second outlet (not shown) for removal of combustion product mixture from the reactor 200, 200′.

Any suitable combustion fuel may be used, including, but not limited to, methane, hydrogen, biogas, natural gas, fossil oil, biomass, or mixtures thereof.

Any suitable combustion catalyst may be used, such as for example, a transition metal and/or a metal oxide, on a support. Suitable examples of the transition metal catalyst include, but are not limited to, platinum (Pt), palladium (Pd), rhodium (Rh), ruthenium (Ru), nickel (Ni), copper (Cu), iron (Fe), cobalt (Co), silver (Ag), iridium (Ir), gold (Au) or their alloys. Suitable examples of the metal oxide include, but are not limited to, cerium oxide (CeO₂), zirconium oxide (ZrO₂), manganese Oxide (MnOₓ), vanadium oxide (V₂O₅), copper oxide (CuO), chromium oxide (Cr₂O₃), Cobalt oxide (CO₂O₄), iron oxide (Fe₂O₃) or mixtures thereof. Any suitable support may be used for the metal and/or the metal oxide, including, but not limited to, alumina, silica, magnesium oxide, barium oxide, strontium oxide, lanthanum oxide, scandium oxide, titanium oxide, vanadium oxide, chromium oxide, manganese oxide, zirconium oxide, niobium oxide, zinc oxide, bismuth oxide or mixtures thereof.

In an aspect of the reactor, the source of process heat comprises a conductive heating element configured to generate the process heat by Joule heating upon passage of electrical current through the conductive heating element. In an embodiment of the reactor, the first surface of the thermal catalytic reactor member on which the NMC catalyst is disposed comprises a surface of the conductive heating element. In another embodiment, the conductive heating element comprises a first member of the thermal catalytic reactor member in thermal communication with a second member of the thermal catalytic reactor member that defines the first surface on which the NMC catalyst is disposed.

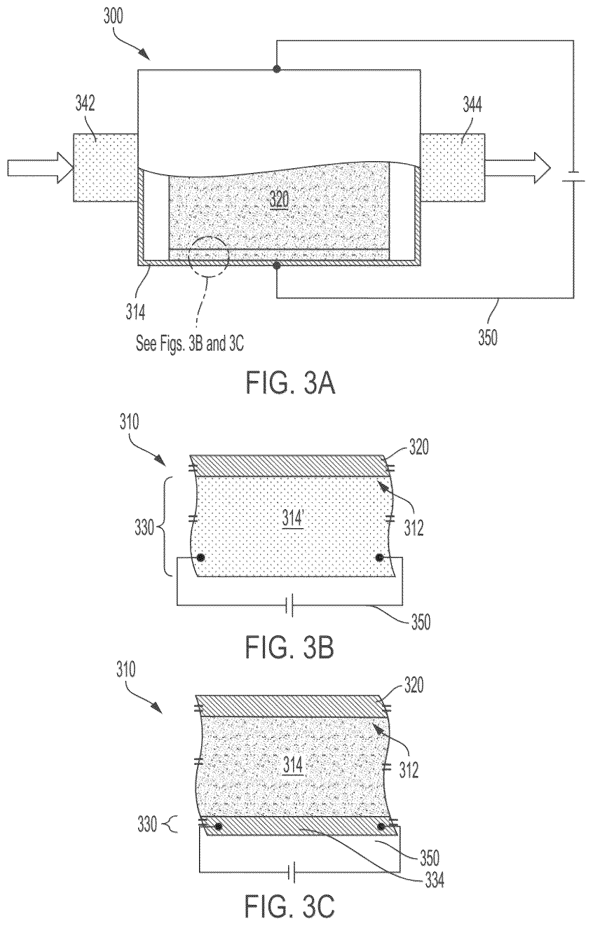

FIG. 3A shows a schematic illustration of an exemplary reactor 300 comprising a thermal catalytic wall reactor member 310 having an NMC catalyst 320 disposed on the inner surface 312 of the wall 314 of the thermal catalytic wall reactor member 310 and a conductive heating element 350 as a source of process heat, according to an embodiment of the present invention.

FIG. 3B shows a schematic illustration of a portion of the exemplary thermal catalytic wall reactor member shown in FIG. 3A, where the wall is configured as a conductive heating element, according to an embodiment of the present invention. The NMC catalyst 320 is coated onto the inner surface 312 of the thermal catalytic reactor tube 310. Both ends of the thermal catalytic reactor tube 310 are connected to an external circuit 350 to heat it up to the reaction temperature (e.g., 900-1100° C.). The NMC feed flows in from one opening 342 and the product stream exits from the other opening 344 of the thermal catalytic reactor tube 310.

FIG. 3C shows a schematic illustration of a portion of the exemplary thermal catalytic wall reactor member 310 shown in FIG. 3A, where the conductive heating element 334 is disposed on the outer side of the wall 314 of and in thermal communication with the thermal catalytic wall reactor member 310, according to an embodiment of the present invention.

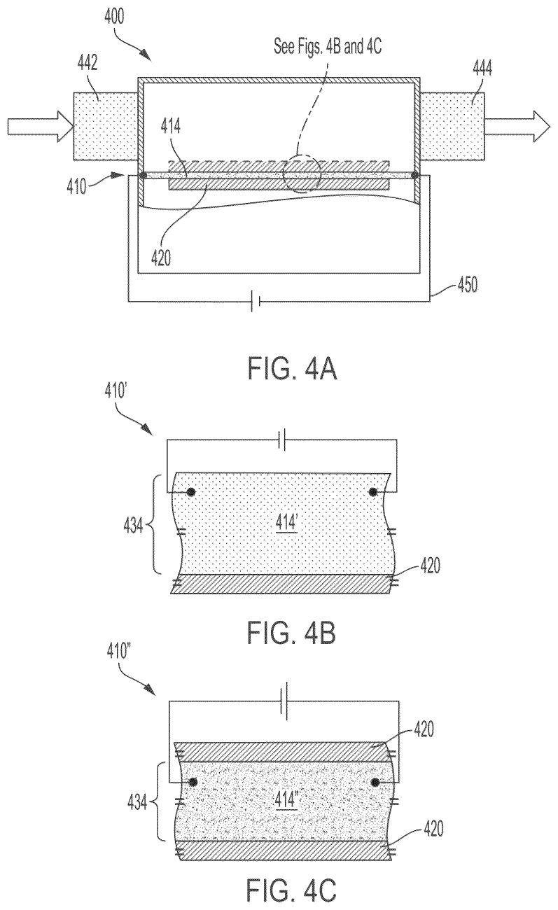

FIG. 4A shows a schematic illustration of an exemplary reactor 400 comprising a thermal catalytic reactor member 410 having (i) an NMC catalyst 420 in the center of the reactor 400 and (ii) a conductive heating element 432 configured to be a source of process heat, according to an embodiment of the present invention. The centrally located conductive heating element 410 may be in any suitable geometry including, but not limited to, one or more of hollow fibers, wires, rods, strips, plates, tubes, meshes, or monoliths. The conductive heating element 410 is connected to an external circuit 450 for heating up to the reaction temperature (e.g., 900-1100° C.). The NMC feed stream is fed in from one opening 442 and the product stream exits from the other opening 444 of the reactor tube 400.

FIG. 4B shows a schematic illustration of a portion of the exemplary thermal catalytic wall reactor member 410′ shown in FIG. 4A, where the NMC catalyst 420 is disposed on one side of a substrate 414′ of the thermal catalytic wall reactor member 410′, according to an embodiment of the present invention.

FIG. 4C shows a schematic illustration of a portion of the exemplary thermal catalytic wall reactor member 410″ shown in FIG. 4A, where the NMC catalyst 420 is disposed on at least two sides of a substrate 414″ of the thermal catalytic wall reactor member 410″, according to an embodiment of the present invention.

Any suitable material may be used for the conductive heating element, including, but not limited to, a conductive ceramic, a metal carbide, a metal nitride, a two-dimensional MXene material, a metal, an alloy, a conductive carbon, or a combination thereof.

In an embodiment, the conductive ceramic comprises mixed metal oxides of perovskite-type oxide conductor represented by a formula of M′Ce_{1-x-y}ZrₓM″_{y}O_{3-δ}, where
M′ is Sr or Ba,
M″ is at least one of Ti, V, Cr, Mn, Fe, Co Ni, Cu, Nb, Mo, W, Pr, Nd, Pm, Sm,
Eu, Gd, Tb, Dy, Ho, Er, Tm and Yb,
x is in the range of 0.1 and 0.2, inclusive,
y is in the range of 0.1 and 0.3, inclusive, and
δ is in the range of 0 to 0.7.

For example, an exemplary perovskite-type oxide conductor can be formed of SrCe_{0.7}Zr_{0.2}Eu_{0.1}O_{3-δ}ceramic. The porous support can be formed of a material having a formula of M′Ce₁₋ₓZrₓO_{3-δ}, where M′ is Sr or Ba, and x is between 0.1 and 0.3, inclusive. For example, an exemplary support can be formed of SrCe_{0.8}Zr_{0.2}O₃.

Suitable metal carbide for use as conductive heating element include, but are not limited to, silicon carbide (SIC), tungsten carbide (WC), Titanium Carbide (TIC), Zirconium Carbide (ZrC), Hafnium Carbide (HfC), Niobium Carbide (NbC), titanium aluminum carbide (Ti₂AlC), and the like.

Suitable metal nitrides for use as conductive heating element include, but are not limited to, titanium nitride (TiN), tantalum nitride (TaN), vanadium nitride (VN), molybdenum nitride (MON), Ti₂NCd, Ti₂NAl, and the like.

Exemplary two-dimensional MXene material for use as conductive heating element include, but are not limited to, Ti₂C, Nb₂C, (Ti_{2-y}Nb_{y})C, Ti₂N, Nb_{1 33}C, and the like.

The metal for use as conductive heating may be selected from the group consisting of tungsten, molybdenum, nickel, copper, chromium, titanium, silver, tungsten, tantalum, and the alloy may be selected from the group consisting of nichrome (Ni₈₀Cr₂O), constantan (CuNi₄₄), manganin (CuMn₁₂Ni), nitinol (NiTi), nichrome-V (Ni₆₀Cr₁₆Si₁₄B), copper-nickel (CuNi) alloy, iron-chromium-aluminum (FeCrAl) alloy, and stainless steel.

Any suitable conductive carbon may be used including, but not limited to, carbon nanotubes, graphene, and graphite.

In an embodiment, the NMC catalyst layer may comprise single metal atoms, sub-nanometer clusters of metals on a support, metal nanoparticles on a support. The metal comprises one or more of Li, Na, K, Mg, Al, Ca, Sr, Ba, Y, La, Ti, Zr, Ce, Cr, Mo, W, Re, Fe, Co, Ni, Cu, Zn, Ge, In, Sn, Pb, Bi, and Mn, and the support comprises quartz, quartz melt, cristobalite, silicate-1 vitreous silica, and zeolite.

In one or more embodiments, the NMC catalyst can comprise metal elements doped (i.e., lattice doping) in the lattice of amorphous-molten-state materials made from Si bonded with one or two of elemental C, N or O, for example, SiO₂. In lattice doping, the dopant metal elements exchange with the lattice elements in the doped materials such that the metal dopant elements are confined in the lattice of the doped materials. For example, the amount of dopant metal can be between 0.001 wt % to 30 wt %, or 0.005 wt % to 20 wt %, or 0.01 wt % to 10 wt % of the total weight of the catalyst. For example, the dopant metal elements can be one or more of Li, Na, K, Mg, Al, Ca, Sr, Ba, Y, La, Ti, Zr, Ce, Cr, Mo, W, Re, Fe, Co, Ni, Cu, Zn, Ge, In, Sn, Pb, Bi, Mn, such as Fe.

In some embodiments, the NMC catalyst comprises a metal doped in a quartz melt phase. In other embodiments, the metal comprises at least one of Li, K, Mg, Al, Ca, Sr, Ba, Ti, Ce, Mn, Zn, Co, Ni, and Fe. In an embodiment, the NMC catalyst comprises Fe@SiO₂, where @ denotes confinement, and characterized by lattice-confined single iron sites embedded within a silica matrix.

In another embodiment, the NMC catalyst comprises metal/zeolite. In yet another embodiment, the metal of the NMC catalyst can be Mo, Re, Zr, Zn, or W, and the zeolite can be MFI (ZSM5), MWW, BEA, or MOR. In the first embodiments or any other disclosed embodiment, the catalyst comprises Mo/ZSM5. Such a catalyst can be created with variable meso-/micro-porosity, with zeolite porosity optimized for methane conversion, for example, using a dual-template assisted synthesis method, an incipient wetness impregnation method, or any other method known in the art.

In an aspect, the reactors as disclosed hereinabove further comprises a hydrogen separation membrane disposed in proximity to the NMC catalyst layer. In an embodiment, the hydrogen separation membranes is coke-resistant by having a pore structure tuned to exclude sorption of one or more coke precursors. In an embodiment, the hydrogen separation membrane comprises one or more tubular membranes. In another embodiment, the hydrogen separation membrane comprises a palladium-metal based membrane, carbon molecular sieve membrane, zeolite membrane, silica membrane, alumina membrane, perovskite oxide membrane, or a mixed metal oxide ceramic membrane.

In various embodiments of the reactor, as disclosed hereinabove, the catalytic reactor member has a geometry selected from the group consisting of tubular, multi-tubular, plate, honeycomb.

### System

In an aspect, there is a system comprising the reactor as disclosed herein above and a first gas flow controller configured to control flow-rate of methane gas through the first inlet over the NMC catalyst.

In some embodiments of the system, where the source of process heat comprises a combustion catalyst, the system may further comprise a second gas flow controller configured to control flow rate and direction of flow of the combustion fuel over the combustion catalyst.

In other embodiments of the system, where the source of process heat comprises a conductive heating element, the system may further comprise a current controller configured to control the amount of electrical current through the conductive heating element.

### Method

In an aspect, there is a method of converting methane non-oxidatively, the method comprising the steps of providing the reactor according to any of the embodiments as disclosed hereinabove. The method also comprises contacting the NMC catalyst layer, in the reaction zone, with methane at a desired flow rate, to produce the product mixture comprising hydrogen and a C₂₊ hydrocarbon. The method further comprises chemically or electrically generating an amount of process heat with the source of process heat to thereby deliver the process heat to the reaction zone on the first surface, such that the process heat creates a temperature in the range of 850° C. to about 1150° C., or 900° C. to 1100° C. at the reaction zone, thereby resulting in a non-uniform temperature profile inside the reactor, where the temperature increases and decreases at a rate in the range of 1 to 200° C./mm, 10 to 150° C./mm, or 30 to 60° C./mm away from the thermal catalytic reactor member.

In an embodiment, the C₂₊ hydrocarbons comprise acetylene, ethylene, ethane, benzene, toluene, naphthalene, or a combination thereof.

The flow rate of methane may be in any suitable range, for example in the range of 5 ml/min to about 500 ml/min, or 5 ml/min to about 250 ml/min, or 5 ml/min to about 100 ml/min, or 5 ml/min to about 50 ml/min, or 5 ml/min to about 25 ml/min.

In an embodiment of the reactor where the source of process heat comprises a combustion catalyst on the second surface, the step of chemically generating heat on the second surface comprises contacting a gas containing oxygen with the combustion catalyst. The method may further comprise controlling one or more of (i) the flow rate of methane and the gas, (ii) direction of flow of the gas to regulate a percentage of methane converted and/or a selectivity of the methane conversion to one or more of the products, and (iii) catalytic wall length.

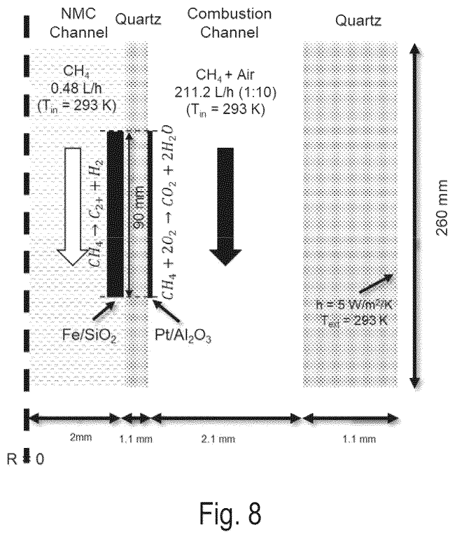

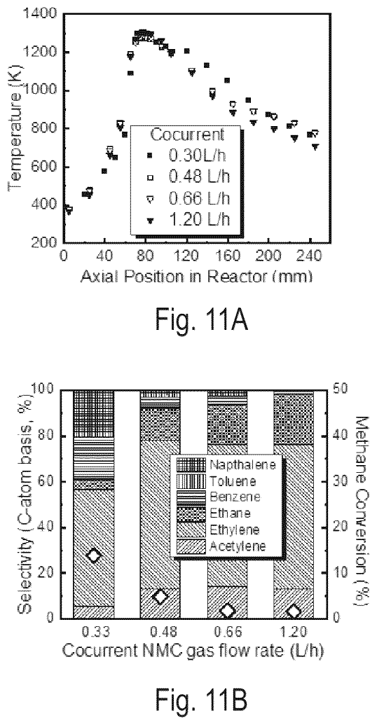

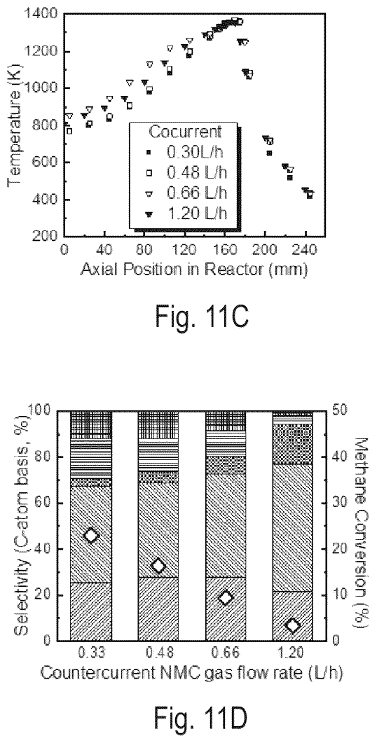

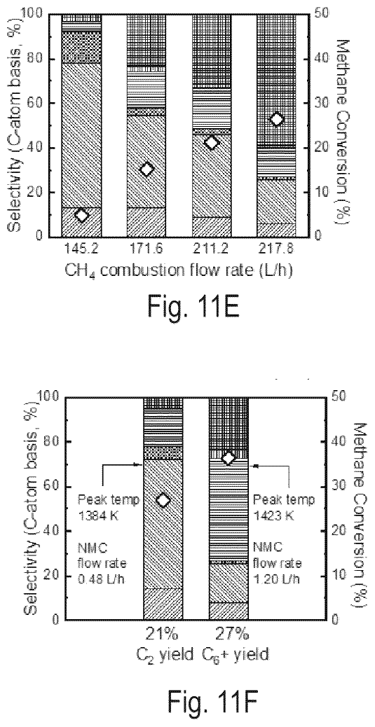

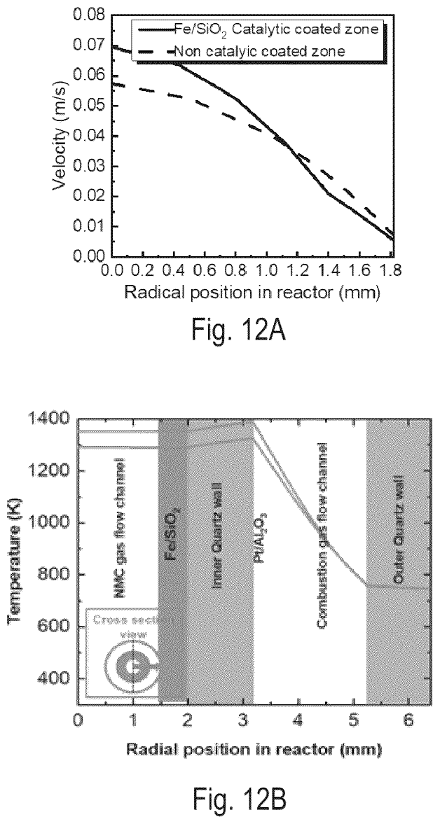

In an embodiment, the direction of flow of methane over the NMC catalyst is the same, i.e. co-current, as the direction of flow of combustion fuel, including air and fuel, over the combustion catalyst, as shown in FIG. 8. In other embodiments, the direction of flow of methane over the NMC catalyst is opposite, i.e. counter-current, to the direction of flow of combustion fuel, including air and fuel, over the combustion catalyst. The effect of the gas flow rate and the direction of flow (co-current versus counter-current) on methane conversion and product selectivity in a reactor according to embodiments of the present invention, is shown in FIGS. 11A-11F and is discussed in detail hereinbelow.

In another embodiment of the reactor where the source of process heat comprises an electrically conductive heating element, the step of electrically generating the process heat comprises passing an electrical current through the conductive heating element. The method may further comprise controlling an amount of electrical current or power to maintain the temperature of the reaction zone to thereby regulate a percentage of methane converted and/or a selectivity of the methane conversion to one or more of the products.

In an embodiment of the method, the percentage of methane converted is greater than 1.5%, or 5% or 10%, or 15%, or 20%, or 25%, or 30%, 35%, or 40%, or 50%, or 60%, or 75%, or 80%, or 90%. In another embodiment, an yield of C₂₊hydrocarbons is greater than 4.5%, or 7.5%, or 10%, or 14%, or 20%, or 25%, or 30%, or 40%, or 50%, or 60%, or 75%, or 80%, or 90%.

In another embodiment, the coke is produced in an amount of 10% or less, or 8% or less, or 6% or less, or 4% or less, or 3% or less, or 2% or less, or 1% or less.

In an aspect, the method further comprises separating hydrogen from the product mixture by a hydrogen separation membrane disposed in proximity to the NMC catalyst layer.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

### Examples

Examples of the present invention will now be described. The technical scope of the present invention is not limited to the examples described below.

### Methods

### Example S1: Fabrication of Autothermal Catalytic Wall Reactor

### S1.1 Reactor Structure

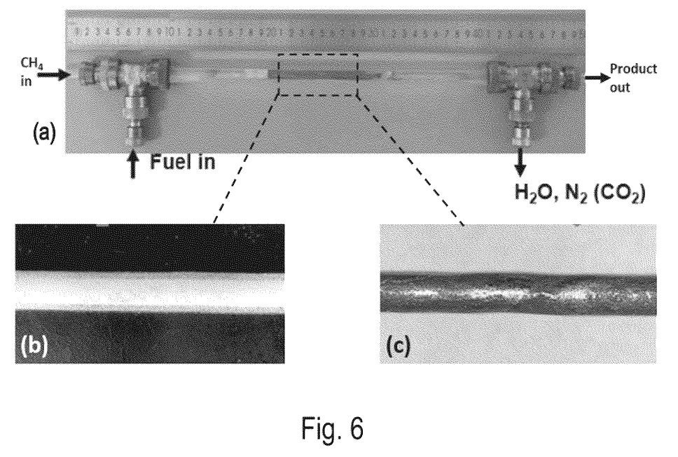

The autothermal millisecond catalytic wall reactor was made of two concentric quartz tubes, as shown in FIG. 6. The inner tube had an outside diameter of 6.35 mm and a thickness of 1.00 mm. The outer tube had an outside diameter of 12.7 mm and a thickness of 1.20 mm. The exothermic methane combustion gases (i.e., CH₄ and air) flew through the annulus region of the reactor, while the CH₄ in endothermic NMC flew through the inner tube. The length of the catalyst coatings on the catalytic wall surface was ˜90.00 mm on both sides. The outer tube was insulated by a ceramic fiber blanket and ceramic cylinder. During the reactions, the heat generated from the catalytic combustion is transferred across the quartz wall to drive the endothermic NMC reaction occurring inside the tube.

### S1.2 Thermal Catalytic Reactor Member Fabrication

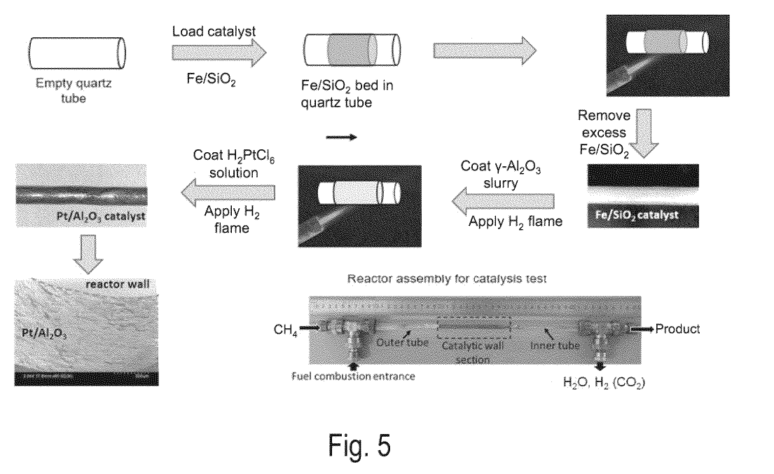

FIG. 5 shows a schematic illustration of an exemplary process of fabricating a thermal catalytic reactor member, where the source of process heat is a combustion catalyst coating, disposed on the outside wall of a tubular thermal reactor member, according to an embodiment of the present invention and further described in sections S1.2.1 and S1.2.2 below.

### S1.2.1 Coating of NMC Catalyst onto the Inner Reactor Wall Surface

The Fe/SiO₂, employed as an NMC catalyst, was prepared as previously reported (Oh et al. “Direct non-oxidative methane conversion in a millisecond catalytic wall reactor.” Angewandte Chemie International Edition 58.21 (2019): 7083-7086; and Liu, Zixiao, et al. “Direct non-oxidative methane coupling on vitreous silica supported iron catalysts.” Catalysis Today 416 (2023): 113873) using a mixture of quartz (SiO₂) and fayalite (Fe₂SiO₄) particles. The quartz particles are commercially available. The Fe₂SiO₄ compound was synthesized through a sol-gel method, as described by DeAngelis et al. (M. T. DeAngelis, A. J. Rondinone, M. D. Pawel, T. C. Labotka, L. M. Anovitz, American Mineralogist 2012, 97, 653-656). The particle mixture underwent ball milling at ambient temperature for 12 hours and was heated to 1700° C. for 6 hours under stagnant air in a high-temperature furnace (Sentro Tech, ST-1700C-101012) or an oxyhydrogen flame to produce Fe/SiO₂. The Fe/SiO₂ catalyst was then crushed and ball milled into a fine powder for the fabrication of the autothermal catalytic wall reactor.

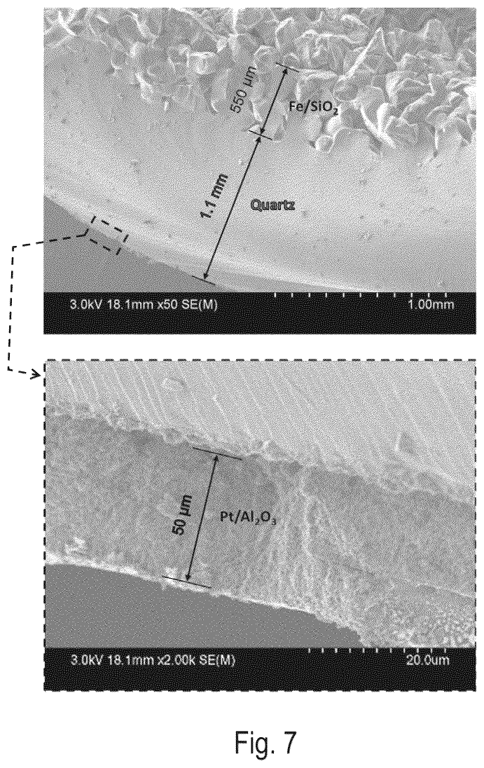

To coat Fe/SiO₂ catalyst onto the inner wall of the quartz tube, sufficient Fe/SiO₂ (0.075 wt % Fe) catalyst was loaded until catalyst length along the tube reached 90.00 mm. The tube was then treated with hydrogen flame repeatedly to ensure uniform dispersion and proper anchoring on the inner wall of the quartz tube. The hydrogen flame was formed by mixing hydrogen (Airgas, industrial grade) and oxygen gas (Airgas, industrial grade) via a hydrogen torch to create a sharp blue flame. By removing the residual catalyst from the tube, a thin uniform layer of Fe/SiO₂ catalyst can be visually observed, as shown in FIG. 6 and FIG. 7. Fe/SiO₂ particles were lightly fused with each other as well as to the quartz wall, forming a 550 μm thick layer.

S1.2.2 Coating of Fuel Combustion Catalyst onto the External Surface of the Reactor Wall

Pt/Al₂O₃ was used as the exothermic reaction catalyst, as a methane oxidation catalyst, was synthesized directly on the external surface of the cylindrical reactor. In order to develop this catalyst layer, an aqueous solution that contained 10.0 wt % γ-alumina (Alfa Aesar, >99.97% purity), 1.0 wt % Cr(NO₃)₃·9H₂O (Alfa Aesar, 99.99% purity), and 0.1 wt % Y (NO₃)₃·6H₂O (Alfa Aesar, 99.9% purity) was firstly prepared. After stirring the mixture for 1 h, the aqueous solution was applied dropwise on the EtOH-cleaned surface on the outside quartz tube, followed by brief treatment with a hydrogen flame. The application of aqueous ceramic mixture onto the reactor tube and flame fusing of the applied materials were repeated until a visible layer of light-yellow coating was formed. This layer is used to improve the adhesion of the fuel combustion catalyst onto the reactor tube. Another 1 g aqueous solution containing 8.0 wt % chloroplatinic acid solution H₂PtCl₆·2H₂O (Alfa Aesar, 99.9% purity) was applied on top of the yellow layer dropwise and treated with a propane torch in a repeating manner. Finally, the washcoat region was exposed to a hydrogen flame one last time to ensure complete decomposition of the metal precursor and secure anchoring of the Pt species. A uniformly dark black washcoat indicated the successful coating of Pt/γ-Al₂O₃ on the quartz reactor. The final catalyst coating was visibly black, as illustrated in FIG. 6 and FIG. 7. The Pt/Al₂O₃ layer formed a continuous layer on the surface of the quartz tube with an average layer thickness of approximately 30 μm.

### Comparative Example 1: Fabrication of a Catalytic Wall Reactor

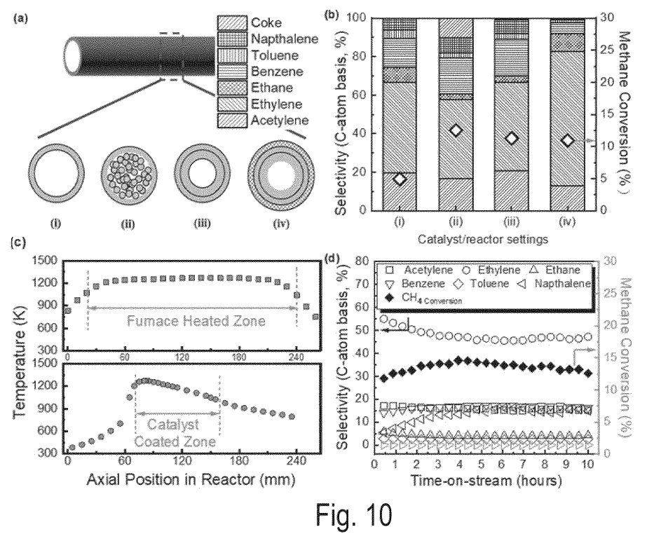

The single layer Fe/SiO₂ catalytic wall reactor was fabricated in a 12.7 mm tube, followed the exact same procedures without the Pt/Al₂O₃ washcoat loading, as shown in FIG. 10(a) (iii). The single layer catalytic wall reactor was operated in conventional electrical furnace. $1.3 NMC reaction test

### Comparative Example 2: S1.3.1 NMC Reaction Test in the Fixed-Bed Reactor

The fixed-bed reactor was made of a non-active straight ¼ inch quartz tube. In the NMC test, 0.375 g of Fe/SiO₂ catalyst was loaded into the reactor, and the position was secured using quartz wool,, as shown in FIG. 10(a) (ii). The reactor was placed horizontally inside a temperature-controlled furnace (Applied Test Systems 3210). A K-type thermocouple was inserted into the reactor to record the temperature. The gas flow was controlled by mass flow controllers (Brooks MFC SLA5850).

Prior to the NMC reaction, the catalyst was heated in an Ar atmosphere (0.48 L/h) to the reaction temperature of 1273 K at 10 K/min ramp rate and kept at the reaction temperature for 1 h. The CH₄ (Airgas MEUHP200, 99.999% purity) and Ar (Airgas ARUHP300, 99.999% purity) gas mixture was then introduced into the reactor at 0.48 L/h (CH₄:Ar=7:1). The product effluents were analyzed online using a gas chromatograph (Agilent Technologies, 5890N) equipped with a DB-WAX column coupled with FID and ShinCarbon ST pack column coupled with TCD to determine methane conversion and hydrocarbon selectivity. To avoid product condensation, all the gas transferring lines were heated to 493 K. The blank reactor test was done using the same conditions as the fixed-bed reactor, except that the catalyst was not used.

### Example S2: NMC Reaction Test in the Autothermal MCWR of Example S1

The catalytic wall reactor was fabricated as described in Example S1 above. To start the autothermal NMC reaction in the reactor, CH₄ (Airgas MEUHP200, purity 99.999%) and Air (Airgas AI B300) in the ratio of 1:10 were fed into the exothermic side, while Ar (Airgas ARUHP300, purity 99.999%) was fed into the endothermic side at room temperature first. The exothermic reaction was ignited by directly flame heating the outer tube over the catalytic wall briefly with a propane torch (Bernzomatic WK2301). The ignited Pt—Al₂O₃ catalyst showed bright red color and extended toward the upstream end of the catalytic wall. The reactor was then insulated by ceramic fibers and cylinders. To monitor the inner tube temperature, a thermocouple was inserted into the inner tube. When the temperature stabilized, CH₄ and Ar with designated flow rates (Ar as internal standard) were fed into the endothermic side. The MCWR was tested at the same conditions as the autothermal MCWR described here, except the reactor was heated by the electric furnace as that in the fixed-bed reactor.

### S2. Residence Time Analysis

### S2.1 Contact Time in Catalytic Wall Reactor

In the present experiment, all gas flows have reynold number lower than 500. In laminar flow, the time for methane to diffuse from the center of the tube to the catalytic surface is defined as contact time. It can be approximately using Eq. (1) below,

$$t ≅ \frac{{x}^{2}}{D} \qquad Eq . ( 1 )$$

where x is the gas transport distance (mm) and D is an approximate gas phase diffusion coefficient (mm2/s). Table S1 lists the calculated results.

TABLE S1
Contact time analysis for reaction gases
in the catalytic wall reactor.

| | Diffusion | Diffusion | Contact |
|---|---|---|---|
| | distance | coefficient¹ | time |
| Cases | (x, mm) | (D, mm²/s) | (t, s) |
| NMC in non-catalytic zone | 2 | 100 | 0.04 |
| NMC in catalytic zone | 1.45 | 100 | 0.02 |
| Methane combustion in non-catalytic | 2.075 | 100 | 0.04 |
| zone | | | |
| Methane combustion in catalytic zone | 2.070 | 100 | 0.04 |

The residence times on both sides of the reactor using inlet room temperature flow can be calculated from:

$$\begin{matrix}{t}_{{res}_{NMC}} ≅ \frac{{V}_{r}}{Flow rate} ≅ \frac{{( \frac{4 mm}{2} )}^{2} * 3.14 * 90 mm}{20 {cm}^{3} / min * 60 s / min} = 3.39 s \\ {t}_{{res}_{me thane combustion}} ≅ \frac{( {( \frac{10.5 mm}{2} )}^{2} - {( \frac{6.35 mm}{2} )}^{2} ) * 3.14 * 90 mm}{3740 {cm}^{3} / min} = 0.0 \; 79 s\end{matrix}$$

The residence time is reduced at high temperatures creating lower gas densities, as discussed in the main text. Nevertheless, the time for diffusion is lower than the minimum experimental residence time of the reactive zone in both the NMC and methane combustion sides.

### S2.2 Residence Time in the Fixed-Bed Reactor

The tortuosity factor of nonporous particles in a packed bed reactor can be modeled using Eq. (2),

$$τ = 1 - pln ( \text{?} = 1 - 0.5 * ln ( 0.4 ) = 146 ≫ 1 \qquad Eq . ( 2 )$$

$$\text{?} \text{indicates text missing or illegible when filed}$$

where τ is the dimensionless tortuosity factor, p is a dimensionless parameter depending on the shape of the particle and their orientation in the bed (0.5), and E is the dimensionless bed void fraction (0.43).

### S2.3 NMC Reaction Product Evaluation Methods

The CH₄ conversion is defined as the percentage ratio of argon normalized converted CH₄ to the argon peak area normalized input CH₄ with argon served as the internal standard. The amount of input CH₄ was determined via GC gas measurement before the reaction occurred.

$${Conversion}_{{CH}_{4}} ( \% ) = \frac{{n}_{{CH}_{4} c onverted}}{{n}_{{CH}_{4} bypass}} * 100$$

The selectivity of a product is defined as the carbon quantity of the product to the converted CH₄.

$${Selectivity}_{{C}_{x} {H}_{y}} ( \% ) = \frac{x * {n}_{{c}_{x} {H}_{y}}}{{n}_{{CH}_{4} c onverted}} * 100$$

The coke selectivity is calculated based on the carbon balance.

$${Selectivity}_{Coke} ( \% ) = 1 - \sum {Selecitivty}_{{C}_{x} {H}_{y}}$$

S3. Energy Balance Analysis for the NMC Coupled with the Fuel Combustion Reaction

The energy balance analysis was performed to explore the heat requirement of the autothermal catalytic wall reactor for NMC. The energy for autothermal operation of the MCWR could be analyzed by calculating the enthalpy of reaction for each product for NMC reaction and methane combustion. The following assumptions were implemented for the energy calculation: (1) coke was assumed to be solid carbon; (2) All hydrocarbons and coke products were assumed to be formed via one-step reaction to simplify the calculation (Eqn 3.1-3.6); (3) The reaction temperature was assumed to be the peak temperature measured; (4) The reactor was adiabatic.

Product formation reactions for the endothermic NMC include:

$${CH}_{4} \to 1 / 2 {C}_{2} {H}_{2} + 3 / 2 {H}_{2} \qquad Eq . ( 3.1 )$$

$${CH}_{4} \to 1 / 2 {C}_{2} {H}_{4} + {H}_{2} \qquad Eq . ( 3.2 )$$

$${CH}_{4} \to 1 / 2 {C}_{2} {H}_{6} + 1 / 2 {H}_{2} \qquad Eq . ( 3.3 )$$

$${CH}_{4} \to 1 / 6 {C}_{6} {H}_{6} + 3 / 2 {H}_{2} \qquad Eq . ( 3.4 )$$

$${CH}_{4} \to 1 / 7 {C}_{7} {H}_{8} + 10 / 7 {H}_{2} \qquad Eq . ( 3.5 )$$

$${CH}_{4} \to 1 / 10 {C}_{10} {H}_{8} + 8 / 5 {H}_{2} \qquad Eq . ( 3.6 )$$

The exothermic methane combustion was:

$${CH}_{4} + 2 {O}_{2} \to {CO}_{2} + 2 {H}_{2} O ( g ) \qquad Eq . ( 4 )$$

The heat of formation of each component at 1 atm and reaction temperature was calculated based on the following equation:

$$Δ {H}_{f} ( T ) = Δ {H}_{f}^{o} + {\int }_{{T}_{o}}^{T} Δ {C}_{p} dt \qquad Eq . ( 5 )$$

$$Δ {H}_{f} ( T ) = Δ {H}_{f}^{o} + {\int }_{{T}_{o}}^{T} a + bT + {cT}^{2} + {dT}^{3} dt \qquad Eq . ( 6 )$$

where ΔH_{f}^{o}(kJ/mol) is the standard enthalpy of formation, ΔCₚ (kJ/mol/K) is the heat capacity of the component, T (K) is the reaction temperature and Tₒ is 298 K, a, b, c and d are the parameters of the heat capacity function of temperature. Table S2 lists the standard enthalpy of formation and heat capacity parameters for each component.

TABLE S2
Standard enthalpy of formation and heat capacity parameters
of reactant and products in the NMC and methane combustion reaction.

| Gas | ${{H}_{f}^{o} ( kJ \cdot {mol}^{- 1} )}^{a}$ | a | b | c | d | e |
|---|---|---|---|---|---|---|
| CH₄ | −74.5 | 19.875 | 5.02E−02 | 1.27E−05 | −1.10E−08 | N/A |
| C₂H₂ | 227.5 | 21.799 | 9.21E−02 | −6.52E−05 | 1.82E−08 | N/A |
| C₂H₄ | 52.5 | 3.95 | 1.56E−01 | −8.34E−05 | 1.77E−08 | N/A |
| C₂H₆ | −83.8 | 6.895 | 1.73E−01 | −6.40E−05 | 7.28E−09 | N/A |
| C₆H₆ | 82.9 | −36.193 | 4.84E−01 | −3.15E−04 | 7.76E−08 | N/A |
| C₇H₈ | 50.10 | 0.29 | 4.7052E−3 | −1.57E−5 | 0 | N/A |
| C₁₀H₈ | 105.6 | 67.099 | 4.32E−2 | 9.17E−4 | −1.00E−6 | 3.09E−10 |
| H₂O | −241.83 | 32.24 | −1.92E−3 | 1.06E−5 | −3.6E−9 | N/A |
| H₂ | 0.00 | 29.088 | −1.92E−3 | 4E−6 | −8.7E−10 | N/A |
| O₂ | 0.00 | 29.11 | −3.86E−6 | 1.75E−6 | −1.07E−8 | N/A |
| CO₂ | −393.5 | 19.8 | 7.34E−2 | −5.6E−5 | 1.71E−8 | N/A |
| Ar | 0 | 20.786 | 2.8259E−7 | −1.464E−7 | 1.092E−7 | −3.66E−8 |
| N₂ | 0 | 31.15 | −1.36E−2 | 2.68E−5 | −1.17E−8 | N/A |

Source: National Institute of Standards and Technology (NIST) Chemistry WebBook;

The heat of the reaction is calculated based on the following equation:

$$Δ {H}_{rxn , i} ( T ) = \sum {v}_{i} Δ {H}_{f , i} ( T ) \qquad Eq . ( 7 )$$

where vᵢ are the stoichiometric coefficients for each component and ΔH_{f,i} is the enthalpy of formation of each component in each reaction, and T (K) is the reaction temperature.

The heat of the NMC and methane combustion reaction was calculated from

$$Δ {H}_{NMC} ( T ) = {Conversion}_{NMC} * \sum {Selecitivity}_{NMCi} * Δ {H}_{rxn , i} ( T ) \qquad Eq . ( 8 )$$

$$Δ {H}_{comb} ( T ) = {Conversion}_{comb} * \sum {Selecitivity}_{comb , i} * Δ {H}_{rxn , i} ( T ) \qquad Eq . ( 9 )$$

The energy required to allow the reactants to reach the reaction temperature can be calculated as

$${Heat}_{NMC .} ( T ) = \sum {f}_{i} {\int }_{{T}_{o}}^{T} Δ {C}_{p , i} dt \qquad Eq . ( 10 )$$

$${Heat}_{comb} ( T ) = \sum {f}_{i} {\int }_{{T}_{o}}^{T} Δ {C}_{p , i} dt \qquad Eq . ( 11 )$$

Here fᵢ is the fraction of reactants and

$$Δ {H}_{f}^{o}$$

(kJ/mol) is the standard enthalpy of formation.
S3.1 Sample Composition Used for the Heat Required for the NMC and the Heat Released from the Methane Combustion

NMC reaction:
Gas flow rate: 1.2 L/h (CH₄:Ar = 9:1)
Reaction temperature: 1423 K

| Methane | Selectivity | | | | | | |
|---|---|---|---|---|---|---|---|
| conversion | Acetylene | Ethylene | Ethane | Benzene | Toluene | Naphthalene | Coke |
| 36.4% | 8.1% | 17.4% | 1.4% | 45.7% | 3.8% | 23.6% | 0% |

Methane combustion reaction:
Gas flow rate: 224.4 L/h (CH₄:Air = 1:10)
Reaction temperature: 1423 K

| Methane | Selectivity |
|---|---|
| conversion | CO₂ |
| 100% | 100% |

### Step I: Calculation of the Enthalpy of the NMC Reaction

For the NMC reaction, the heat required is calculated as

$$\begin{matrix}Δ {H}_{rxn , NMC} ( 1423 K ) = 36.4 \% * \sum 8.1 \% * 2. * {10}^{2} + 17.4 \% * 1.1 * \\ {10}^{2} + 1.4 \% * 3.9 * 10 + 45.7 \% * 8.5 * 10 + \\ 3.8 \% * 6.3 * 10 + 23.6 \% * 9.7 * 10 \\ = 36.5 kJ / mol {CH}_{4}\end{matrix}$$

### Step II: Calculation of the Enthalpy of the Methane Combustion Reaction

For methane combustion reaction, the heat released can be calculated as

$$\begin{matrix}Δ {H}_{rxn , comb} ( 1423 K ) = 100 \% * \sum 100 \% * ( - 7.9 * {10}^{2} ) \\ = - 790 kJ / mol\end{matrix}$$

### Step III: Calculation for the Heat Requirement for Reactants to Reach the Reaction Temperature

For every mole of the feed gas,

$$\begin{matrix}{Heat}_{NMC} ( T ) = \sum \frac{7}{8} {\int }_{{T}_{o}}^{T} ( 19.875 + 5.02 * 1 {0}^{- 2} T + 1.27 * 1 {0}^{- 5} {T}^{2} - \\ 1.1 * 1 {0}^{- 8} {T}^{3} ) dt + \frac{1}{8} {\int }_{{T}_{o}}^{T} ( 19.875 + 5.02 * 1 {0}^{- 2} T + \\ 1.27 * 1 {0}^{- 5} {T}^{2} - 1.1 * 1 {0}^{- 8} {T}^{3} ) dt \\ = - 790 kJ / mol\end{matrix}$$

$$\begin{matrix}{Heat}_{comb} ( T ) = \sum \frac{1}{11} {\int }_{{T}_{o}}^{T} ( 19.875 + 5.02 * 1 {0}^{- 2} T + 1.27 * 1 {0}^{- 5} {T}^{2} - \\ 1.1 * 1 {0}^{- 8} {T}^{3} ) dt + \frac{2}{11} {\int }_{{T}_{o}}^{T} ( 28.11 - 3.86 * 1 {0}^{- 6} T + \\ 1.75 * 1 {0}^{- 6} {T}^{2} - 1.07 * 1 {0}^{- 8} {T}^{3} ) dt + \frac{8}{11} {\int }_{{T}_{o}}^{T} ( 31.15 - \\ 1.36 * {10}^{- 2} T + 2.68 * 1 {0}^{- 5} {T}^{2} - 1.17 * 1 {0}^{- 8} {T}^{3} ) d t \\ = 34 kJ / mol\end{matrix}$$

Step IV: Calculation of the Overall Heat Requirement for the NMC Reaction and the Heat Released from the Methane Combustion Reaction at 1423 K.

The overall heat requirement for the NMC reaction includes the heat required to increase the reactant gas mixture (CH₄ and Ar) to 1423 K and the heat of the NMC reaction.

$$Δ {H}_{NMC} ( 1423 K ) = Δ {H}_{rxn , NMC} ( 1423 K ) + {Heat}_{NMC} ( 1423 K ) = 114.5 kJ / mol$$

$$n Δ {H}_{NMC} ( 1423 K ) = 1 * 1 {0}^{- 2} mol / h * 116.5 kJ / h = 1.2 kJ / h$$

The overall heat released from the methane combustion reaction includes the heat to increase the reactant gas mixture (CH₄ and Air) to 1423 K and the heat of the methane combustion reaction.

$$Δ {H}_{Comb} ( 1423 K ) = Δ {H}_{rxn , comb} ( 1423 K ) + {Heat}_{comb} ( 1423 K ) = - 756 \text{kJ/} mol$$

$$n Δ {H}_{Comb} ( 1423 K ) = 1.9 mol \text{/h} * ( - 7 \; 56 \text{kJ/} mol ) = - 1 \; 453 \text{kJ/h}$$

Therefore, the heat release of methane combustion is far more than the heat energy required by the NMC reaction to sustain the autothermal operation. This has also indicated majority of the heat was lost during the experiment, something expected for small-scale devices and a better insulation design is required to prevent such heat losses.

### S3.2 H₂ Combustion as an Alternative Energy Source

In order to achieve the goal of zero greenhouse gas emission, H₂ combustion was analysed to couple with the NMC reaction. The combustion was set to the reaction stoichiometric ratio, where H₂: Air flow ratio is 1:2.5.

$$\begin{matrix}Δ {H}_{Comb , {H}_{2}} ( 1423 K ) = Δ {H}_{rxn , comb , {H}_{2}} ( 1423 K ) + {Heat}_{c o m b , {H}_{2}} ( 1423 K ) \\ = ( - 46 6.7 \text{kJ/} mol ) + 31 \text{kJ/} mol = - 4 35.7 \text{kJ/} mol\end{matrix}$$

If the reaction operated under adiabatic conditions, the H₂ mole flow rate (n_{H}_{₂}) required for combustion reaction to substain autothermal operation is

$${n}_{{H}_{2}} = \frac{1}{3.5} * \frac{1.2 \text{kJ/h}}{4357 \text{kJ/} mol} = 7.9 * 1 {0}^{- 4} mol \text{/h}$$

Based on the experimental hydrogen yield and methane mole flow rate, the H₂ produced rate (n_{NMC,H}_{₂}) from NMC reaction at 1423 K is determined to be

$${n}_{NMC , {H}_{2}} = 43.4 \% * 0.0103 mol \text{/h} = 4.5 * 1 {0}^{- 3} mol \text{/h}$$

Thus, the percentage of H₂ generated from NMC reaction for suistaining combustion reaction can be determined as

$$\% {H}_{2} required from NMC = \frac{7.9 * 1 {0}^{- 4} mol \text{/h}}{4.5 * 1 {0}^{- 3} mol \text{/h}} * 100 = 17.6 \%$$

### S4. COMSOL Simulation

COMSOL Multphysics R, a general-purpose simulation software, is referred to herein as COMSOL. 2D-axisymmetric steady-state COMSOL simulations were performed to understand the effects of gas flow velocities and temperature profile along the radial direction after coupling with NMC and methane combustion reaction.

FIG. 8 shows the model geometry of the autothermal MCWR.

The following assumptions were made:
1. No slip-conditions are used for flow modeling, with symmetry condition imposed at R=0.
2. All gas flows enter the reactor at room temperature, with an open boundary condition at the outlet allowing for gas and heat to be carried away from the modeling domain.
3. The thin Pt/Al₂O₃ layer for methane combustion reaction has little to no effect on the gas flow velocity profile, therefore it is treated as a boundary in simulations.
4. For thermal modeling, the heat of combustion of methane increases the temperature in the combustion chamber. Due to the lack of simplified reaction kinetics for CH₄ combustion reaction, especially under sudden temperature ramping and cooling, the heat flux is assumed to be uniform over the surface area of the catalyst (and hence the entire axial length of the catalyst zone) and determined using the overall methane conversion (i.e., 100%) and inlet flow rates.
5. A wavelength-independent radiative emissivity of 0.65 and convective heat transfer coefficient of 5 W/m²/K are used at the outer environment boundaries to account for heat exchange between surfaces and losses to the ambient. These are based on typical values encountered in the literature², and deemed reasonable owing to the complexity in determining these quantities precisely.

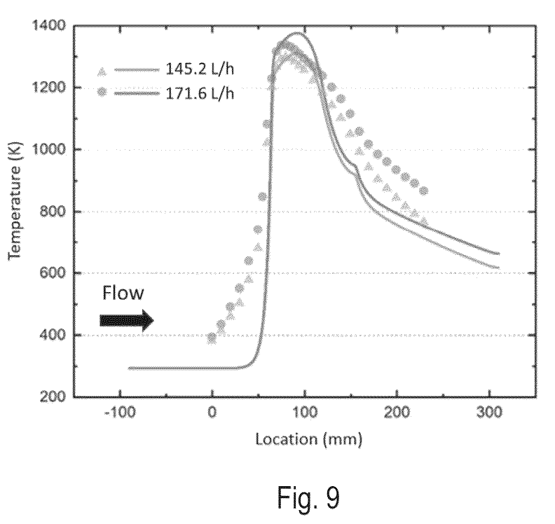

Under these assumptions, the model of the temperature profile in the NMC reaction chamber arising from complete combustion over the whole of the catalyst layer and find a reasonable agreement with experimental measurements, as indicated in FIG. 9. The model does not capture the broadening of the temperature profile around peak temperatures, potentially arising due to radiative transport heating surfaces upstream and downstream. Regardless, in physical agreement with experiments, higher peak temperatures are observed with a higher flow rate as complete methane combustion is assumed, leading to larger heat of reaction. Notably, the model qualitatively agrees with the experimental hypothesis that the asymmetry of temperature profile around the peak underlies the autothermal MCWR reactor. In counter-current flow, a rapid quenching is observed post-peak temperature in the reaction zone. In contrast, when the flow is reversed (i.e., co-current), the quenching will be slower.

### Experiment 1: Nonoxidative Methane Coupling in an Autothermal Millisecond Catalytic Wall Reactor (MCWR)

### SUMMARY

Direct non-oxidative methane coupling (NMC) is as an important one-step pathway for upgrading methane to value-added larger hydrocarbons and hydrogen. The issues of low methane conversion, low selectivity to lighter hydrocarbons, and poor catalyst durability are rooted in the endothermicity of methane activation requiring high temperatures with subsequent promotion of heavy hydrocarbon and coke-forming reaction. Disclosed herein is a millisecond catalytic wall reactor that resolves these challenges with thermal self-sustainability. The engineered reactor, coated with NMC and combustion catalysts on opposite tube wall surfaces, enables milli-second activation of methane powered by the energy generated from catalytic combustion of fuel gas with greatly improved heat transfer by removing the thermal boundary layers and controlled reaction zone with sharp temperature gradient, which inhibits the heavy product or coke formation. This reactor innovation leads to unprecedented NMC performance with high single-pass CH₄ conversion, >99% C₂₊ selectivity, negligible coke formation, and autothermal operation.

### Experimental Procedure

The autothermal MCWR was fabricated by flame fusion of a Fe/SiO₂ catalyst into the inner wall of a quartz reactor. The Pt/Al₂O₃ catalyst layer was developed by flame fusing the outer surface of the reactor wall that was pre-coated with an Al₂O₃ precursor, followed by coating with the Pt precursor and then flame treating. The experimental details on catalyst synthesis and reactor fabrication are described above in Example S1, and the reactor operation and NMC reaction tests, reaction result analysis, COMSOL Multiphysics R simulation details, and process simulation of the MCWR reactor are described in the Example S2 above.

### Results and Discussion

In this experiment, motivated by the potential of improving NMC performance by engineering the thermal driving force, the inventors designed, constructed, and operated a modular autothermal millisecond catalytic wall reactor (MCWR). The exemplary autothermal reactor (MCWR), according to embodiments of the present invention, uses well-known NMC catalyst, and yet is able to push the performance to generate unprecedently high methane conversion, hydrocarbon product selectivity, and maintain catalyst durability. The developed autothermal MCWR reactor (schematic in FIG. 2B, is made of a quartz tube, coated with NMC and fuel combustion catalysts on opposite tube wall surfaces (see Example S1). The NMC catalyst induces CH₄ activation by heterogeneous surface dehydrogenation at a medium-high temperature (1173-1523 K) that is maintained by the fuel combustion on the other side of the reactor wall using the fed CH₄/air mixture. Placing the catalysts directly onto the inner (Fe/SiO₂ for endothermic NMC) and outer (Pt/Al₂O₃ for exothermic combustion) walls of reactor promotes heat transfer in thermal boundary layers. Sharp temperature gradients along axial direction controlled by catalyst zoning, together with short contact-time (e.g., diffusion time of reactant to the reactive wall is within millisecond), inhibit the secondary and follow-on reactions avoiding coke formation and catalyst deactivation enabling excellent performance.

The capability of the exemplary autothermal MCWR, according to embodiments of the present invention, is established by comparison to three controls—a blank reactor, a fixed-bed reactor packed with the Fe/SiO₂ catalyst, and a MCWR coated with the Fe/SiO₂ catalyst, that were all heated by an electric furnace (FIG. 10(a)). The CH₄ conversions were 4.9% (blank), 12.5% (fixed-bed), 11.3% (MCWR) and 11.0% (autothermal MCWR) (FIG. 10(b)), with the peak reactor temperature controlled at 1273 K across all cases (FIG. 10(c)). Reactor axial position “0 mm” is defined as the gas inlet of the NMC reaction. The presence of Fe/SiO₂ catalyst increased CH₄ conversion (blank versus rest), confirming the existence of heterogeneous methane activation, although it is not sensitively influenced by the catalyst loading quantity and format, as the fixed-bed reactor and MCWR showed similar conversions. The product selectivity, however, is significantly influenced by the reactor heating and catalyst loading methods (FIG. 10(b)). The coke selectivity shifted from 10.2% in the fixed-bed reactor to 0.8% in the MCWR heated by electric furnace to ˜0 in the autothermal MCWR. Importantly, the C2 selectivity increased favorably from 60.8% and 69.9% to 92.2% in sequence, accompanied with a decrease in aromatics selectivity from 28.9% and 29.4% to 7.8%. NMC over the Fe/SiO₂ catalyst has a mixed heterogeneous-homogeneous reaction network, in which the catalyst initiates CH₄ dehydrogenation, generating methyl and hydrogen species, followed by a series of subsequent reaction steps that form dehydrogenated and cyclized larger hydrocarbon products. The short-contact-time in the MCWR to suppress subsequent chemistries is an important aspect in enhancing the olefin and hydrogen production. However, this characteristic alone is not sufficient to extensively suppress coke formation in NMC. Combining the short residence time with the rapid thermal activation and quenching zones developed in the autothermal MCWR (FIG. 10(c)), in contrast to the uniform isothermal zone in electrically heated reactors heated, is a key factor in inhibiting secondary and follow-on reactions. This inhibition underlies the observed high C2 selectivity and negligible coke formation. Furthermore, such an exceptional performance is found to be stable over the whole duration of a 10h operation (FIG. 10(d)). Post an initial induction period (˜2 h, caused when NMC flow switched from inert gas to methane, see section S1.3), the time-on-stream (TOS) performance reveals a stable reaction with high methane conversion (˜13.7%), high C₂₊ hydrocarbons selectivity (66.4% C2 and 33.6% aromatics), and negligible coking.

Next, to further tune these performance metrics and temperature profiles, the relative flow direction (i.e., co-current versus counter-current flow) and gas flow rates in the reaction channel and combustion channel were studied independently. In the co-current flow mode, under a fixed CH₄/air flow in the exothermic fuel combustion channel, the increase in CH₄ flow in the endothermic NMC channel decreased the contact time of CH₄ (section Example S1) as well as the width and peak temperature of the heating zone (FIG. 11A). The methane conversion in the NMC channel decreased from 14.2% to 1.6% while the C2 selectivity increased from 61.9% to 98.0% at the expense of aromatics (FIG. 11B). Counter-current CH₄/air flow similarly impacted the temperature profiles (peak temperature, breadth of heating zone, see (FIG. 11C) and NMC performance (methane conversion, product selectivity, see (FIG. 11D) as the CH₄ flow in the NMC reaction channel increased. A comparison between co-current and counter-current flow modes reveals that the peak temperature and its position were 59, 96, 90, and 60 K higher and ˜90 mm later in the counter-current cases for the 0.3, 0.48, 0.66 and 1.2 L/h NMC flow rate, respectively. These led to higher CH₄ conversions, higher acetylene and lower aromatics selectivity than the co-current flow mode at the same flow rates in both reaction channels. The increase in CH₄ conversion could be attributed to its longer passage to reach the peak temperature and the increased peak temperature in counter-current mode. The enhancement in acetylene selectivity was attributable to the very sharp temperature drop after the maximum temperature. The decrease in aromatics selectivity indicates that acetylene is the precursor of benzene and other aromatics, as the sudden temperature drop (or cooling) after the CH₄ activation zone limited the secondary reaction steps in NMC. Further studies in the co-current flow mode showed that the NMC methane conversion increased with an increase of CH₄/air flow in the combustion channel under fixed flow rate in the NMC reaction channel (FIG. 11E), contributed by a higher peak temperature associated with higher heat energy released from CH₄ combustion. The naphthalene selectivity increased with increased methane conversion could be contributed to the elevated temperature profile, which would also promote secondary gas phase reaction in the catalytic combustion zone. The manipulation of gas flow rates in both reaction channels under co-current flow pattern promoted CH₄ conversion while tuned the selectivity either towards ethylene or benzene (FIG. 11F), and a single pass ˜37.0% CH₄ conversion, >99.0% C₂₊ selectivity and negligible coke were achieved in NMC in the autothermal MCWR with 1.2L/h NMC gas flow rate and peak temperature at 1423 K under co-current operation.

The gas velocity profile and radial temperature could influence the performance of MCWR process, but they were difficult to measure accurately in experiments due to the small diameter of the reactor tube. A reactor model was developed using COMSOL Multiphysics (see section S4 for model details) to reveal this information on the basis of reactor dimensions and operating conditions. First, the flow was simulated in the NMC channel to understand the time-scales that enable efficient catalytic performance. In the NMC channel (2 mm in diameter), the effective flow region was reduced as the catalyst coating thickness was changed (FIG. 12A). The simulation indicated that the maximum velocity at the high temperatures in the NMC chamber could increase from ˜100 mm/s to 200 mm/s when the catalyst coating thickness is changed from 0.2 mm to 0.55 mm (used in the experimental study). Thus, at this experimental coating thickness, the residence time over the catalytic length (˜90 mm) could be as low as ˜0.5 s while the time required for a reactant methane molecule to diffuse to the catalytic layer surface is an order of magnitude smaller (see SI, section S2.1 for a sample calculation), enabling sufficient contact for NMC reaction to proceed without diffusion limitation. Additionally, the reduction of the contact time in the NMC layer impacted the maximum gas temperature, with the model predicting a decrease of peak temperature with an increase in catalyst coating thickness. Future designs to enable the maximum throughput from the proposed modular system by optimizing the diameter of the NMC chamber in conjunction with catalyst layer thickness could be envisioned. On the fuel combustion side, the velocity profile was less sensitive to the catalyst coating region around its experimental value owing to its small thickness (˜0.03 mm; result not shown). However, the radial temperature profiles depend on the flow rates in the combustion channel (FIG. 12B). At a higher flow rate of methane, assuming a complete conversion, higher peak temperatures were observed.

Specifically, at 145.2 L/h flow, the Pt/Al₂O₃ catalyst layer had the highest temperature of ˜1310 K. A temperature drop towards the NMC channel direction occurs as heat is conducted through quartz and silica (thermal conductivity K˜1.5 W/m−K). The temperature drops to ˜1295 K upon reaching the reactor's centerline, where a 0.48 L/h flow of Ar is flowing. This NMC temperature could be lower if the endothermicity of the reaction is accounted for. In contrast to the NMC chamber, a sharp temperature drop along radial direction in the combustion reaction channel towards the outer shell was caused by the strong convective and radiative heat losses from catalytic reactor walls. Minimizing these losses using insulation and shielding are potential pathways to maximize system efficiency. It should be noted that adiabatic operation of the MCWR only required 0.18 L/h CH₄/air flow (CH₄: air ratio of 0.1) for fuel combustion to supply heat to the 1.2 L/h CH₄/Ar flow (CH₄:Ar ratio of 9) in NMC channel. This case was used to achieve 36.4% methane conversion, as shown earlier in FIG. 11F. Overall, it was evident from simulations that the close contact between the endothermic NMC and exothermic fuel combustion reactions enabled by catalyst coatings on opposite sides of a reactor wall effectively promotes heat transfer to achieve high temperature for methane activation.

Besides C₂₊ products, NMC generates plenty of H₂ co-product, which could be used as the fuel for the combustion reaction in the autothermal MCWR. Our initial study demonstrated the success of NMC in achieving high C₂₊ yield and negligible coke enabled by the methane fuel combustion over Pt/Al₂O₃ wash-coated catalyst on the reactor wall. However, it cannot be denied that methane combustion carbon emissions via CO₂ production. The replacement of CH₄ by H₂ co-product from NMC would eliminate these carbon emissions while retaining the autothermal operation. To demonstrate this potential, an energy balance calculation for the coupled NMC and H₂/air fuel combustion reactions in MCWR was conducted with methane conversion and product selectivity data from high benzene yield shown in FIG. 11F. Under adiabatic conditions, the combustion of 17.6% H₂ co-product generated from NMC is sufficient to sustain the NMC reaction (section S3.2). A system-level framework analysis suggests the >25% CH₄ conversion and <20% coke selectivity is viable to realize the economic feasibility of the NMC process. The autothermal NMC MCWR surpasses all these performance metrics, indicating strong technoeconomic viability.

### CONCLUSION

In summary, an exemplary autothermal millisecond catalytic wall reactor, according to embodiments of the present invention, self-sustains NMC with high hydrocarbon product selectivity and negligible coke formation. The reactor was made of a quartz tube with NMC and fuel combustion catalyst coatings on opposite sides of the tube wall. The intimate contact between the catalysts and reactor wall enables efficient heat transfer from the exothermic fuel combustion to the endothermic NMC, offering high local temperature at active sites for methane activation. The catalyst zoning in the fuel combustion channel enables a sharp temperature gradient and narrow isothermal zone that effectively suppresses the secondary and follow-on reactions and thus coke formation. By tuning the gas flow rate in each reaction channel and the flow direction, the axial temperature profile was modulated to tune the methane conversion and product selectivity towards C2 or aromatic products. By optimizing the gas flow rates in both channels, high methane conversion, as well as tunable hydrocarbon selectivity, was achieved. The system could potentially be driven by combusting the sole co-product H₂ to create a self-sustained operation of NMC without greenhouse emissions.

Experiment 2: Electrified Catalytic Wall Reactor for Decarbonized Ethylene Production from Renewable Natural Gas

Joule-heated reactor wall with wash-coated catalyst for high-performance NMC. As shown in Experiment No. 1, the structure and activity of M/V—SiO₂ catalysts can be controlled to realize coke resistance and high-temperature durability. The V—SiO₂ confines the metal clusters or single atoms in the structural defects, enhancing metal dispersion and active site stability. Previous studies have demonstrated that Fe/V—SiO₂ outperforms Fe/Cristobalite catalyst in NMC. The inventors manufactured the catalytic wall reactor tube with a spatially controlled catalyst zone consisting of M/V—SiO₂ catalysts wash-coated onto the inner surface of the reactor tube. The direct surface heating of the reactor wall led to millisecond methane activation and a sharp temperature profile within the reactor's flow channel, thus suppressing gas phase reaction and coke formation. Without wishing to be bound by any particular theory, it is believed that the Joule heating will replace fossil-fuel-based heating to decarbonize C₂H₄ production in NMC. The conductive ceramic, metal or alloy will wrap outside of or replace the quartz wall for Joule heating.

Although the invention is illustrated and described herein with reference to specific embodiments, conductive to the details shown. Rather, various modifications may be made in the details within the scope and range of equivalents of the claims and without departing from the invention.

1. A reactor comprising:
a) a thermal catalytic reactor member comprising:
i. a non-oxidative methane coupling (NMC) catalyst disposed on a first surface of a substrate, wherein the NMC catalyst is configured to endothermically convert methane in a reaction zone on the NMC catalyst side of the thermal catalytic reactor member to a product mixture comprising hydrogen and a C₂₊ hydrocarbon, and
ii. a source of process heat configured to deliver heat to the reaction zone by thermal conduction through the thermal catalytic reactor member;
b) a first inlet for contacting the NMC catalyst with methane gas; and
c) a first outlet for removal of product mixture from the reactor.

2. The reactor according to claim 1, wherein the source of process heat comprises one or more of:
i. a combustion catalyst configured to generate the process heat by an exothermic combustion reaction in the presence of a combustion fuel; and
ii. a conductive heating element configured to generate the process heat by Joule heating upon passage of electrical current through the conductive heating element.

3. The reactor according to claim 2, wherein the thermal catalytic reactor member comprises one or more substrates defining the first surface and a second surface disposed on opposite sides of the one or more substrates and isolated from the NMC catalyst layer, and
wherein the combustion catalyst is disposed on the second surface of the one or more substrates.

4. The reactor according to claim 3, wherein the reactor includes a reactor housing having a tubular configuration defined by the one or more substrates forming one or more walls for containing the methane gas, the first surface comprises an inner surface of the one or more walls, and the second surface comprises the outer surface of the one or more walls.

5. The reactor according to claim 2, wherein the reactor includes a reactor housing comprising a tubular reactor defined by one or more inner channels for contacting the combustion fuel with the combustion catalyst disposed on the inner surface of the tubular reactor housing and the NMC catalyst is disposed on the outer surface of the tubular reactor.

6. The reactor according to claim 2, wherein the combustion catalyst comprises a transition metal and/or a metal oxide, on a support.

7. The reactor according to claim 5, wherein the transition metal catalyst comprises platinum (Pt), palladium (Pd), rhodium (Rh), ruthenium (Ru), nickel (Ni), copper (Cu), iron (Fe), cobalt (Co), silver (Ag), iridium (Ir), gold (Au) or their alloys,
wherein the metal oxide comprises cerium oxide (CeO₂), zirconium oxide (ZrO₂), manganese Oxide (MnOₓ), vanadium oxide (V₂O₅), copper oxide (CuO), chromium oxide (Cr₂O₃), Cobalt oxide (Co₂O₄), iron oxide (Fe₂O₃) or mixtures thereof, and wherein the support comprises alumina, silica, magnesium oxide, barium oxide, strontium oxide, lanthanum oxide, scandium oxide, titanium oxide, vanadium oxide, chromium oxide, manganese oxide, zirconium oxide, niobium oxide (Nb₂O₅), zinc oxide, bismuth oxide or mixtures thereof.

8. The reactor according to claim 2, wherein the combustion fuel comprises methane, hydrogen, biogas, natural gas, fossil oil, biomass, oxygen-containing compound, or mixtures thereof.

9. The reactor according to claim 2, wherein the source of process heat comprises the conductive heating element, and wherein (a) the first surface of the thermal catalytic reactor member on which the NMC catalyst is disposed comprises a surface of the conductive heating element, or (b) the conductive heating element comprises a first member of the thermal catalytic reactor member in thermal communication with a second member of the thermal catalytic reactor member that defines the first surface on which the NMC catalyst is disposed.

10. The reactor according to claim 9, wherein the conductive heating element is in a geometry of one or more hollow fibers, wires, rods, strips, plates, tubes, meshes, or monoliths.

11. The reactor according to claim 10, wherein the reactor includes a reactor housing having a tubular configuration defined by one or more sidewalls for containing the methane gas, and wherein the conductive heating element is disposed inside the reactor housing and is isolated from the one or more sidewalls.

12. The reactor of claim 11, wherein the catalytic reactor is tubular reactor having a cylindrical geometry defining a longitudinal axis and the conductive heating element is disposed along the longitudinal axis.

13. The reactor according to claim 1, wherein the C₂₊ hydrocarbons comprises one or more of acetylene, ethylene, ethane, benzene, toluene, naphthalene, and coke.

14. The reactor according to claim 2, wherein the conductive heating element comprises a conductive ceramic, a metal carbide, a metal nitride, a two-dimensional MXene material, a metal, an alloy, a conductive carbon, or a combination thereof.

15. The reactor according to claim 14, wherein the conductive ceramic comprises mixed metal oxides of perovskite-type oxide conductor represented by a formula of M′Ce_{1-x-y}ZrₓM″_{y}O_{3-δ},
where
M′ is Sr or Ba,
M″ is at least one of Ti, V, Cr, Mn, Fe, Co Ni, Cu, Nb, Mo, W, Pr, Nd, Pm, Sm, Eu, Gd,
Tb, Dy, Ho, Er, Tm and Yb,
x is in the range of 0.1 and 0.2, inclusive,
y is in the range of 0.1 and 0.3, inclusive,
δ is in the range of 0 to 0.7;
wherein the metal carbide comprises silicon carbide (SiC), tungsten carbide (WC), Titanium Carbide (TIC), Zirconium Carbide (ZrC), Hafnium Carbide (HfC), Niobium Carbide (NbC), titanium aluminum carbide (Ti₂AlC), and the like;
wherein the metal nitride comprises titanium nitride (TIN), tantalum nitride (TaN), vanadium nitride (VN), molybdenum nitride (MoN), Ti₂NCd, Ti₂NAl, and the like;
wherein the two-dimensional MXene material comprises Ti₂C, Nb₂C, (Ti_{2-y}Nb_{y})C, Ti₂N, Nb_{1.33}C, and the like;
wherein the metal selected from the group consisting of tungsten, molybdenum, nickel, copper, chromium, titanium, silver, tungsten, tantalum; and
wherein the alloy selected from the group consisting of nichrome (Ni₈₀Cr₂O), constantan (CuNi₄₄), manganin (CuMn₁₂Ni), nitinol (NiTi), nichrome-V (Ni₈₀Cr₁₆Si₁₄B), copper-nickel (CuNi) alloy, iron-chromium-aluminum (FeCrAl) alloy, and stainless steel.

16. The reactor according to claim 1,
wherein the NMC catalyst layer comprises single metal atoms, sub-nanometer clusters of metals on a support, metal nanoparticles on a support,
wherein the metal comprises one or more of Li, Na, Mg, Al, Ca, Sr, Ba, Y, La, Ti, Zr, Ce, Cr, Mo, W, Re, Fe, Co, Ni, Cu, Zn, Ge, In, Sn, Pb, Bi and Mn and/or wherein the support comprises quartz melt, cristobalite, silicate-1 vitreous silica, and zeolite.

17. (canceled)

18. The reactor according to claim 1, further comprising a hydrogen separation membrane disposed in proximity to the NMC catalyst layer, wherein the hydrogen separation membrane has a pore structure tuned to exclude sorption of one or more coke precursors and/or comprises one or more of a hollow fiber membrane, a carbon molecular sieve membrane or a mixed metal oxide ceramic membrane.

19. (canceled)

20. (canceled)

21. (canceled)

22. The reactor according to claim 2, further comprising:
i. a second inlet for contacting a combustion fuel over the combustion catalyst; and
ii. a second outlet for removal of combustion product mixture from the reactor.

23. A system comprising:
i. the reactor according to claim 1;
ii a first gas flow controller configured to control flow-rate of methane gas through the first inlet over the NMC catalyst,
iii. a second gas flow controller configured to control flow rate and direction of flow of the combustion fuel over the combustion catalyst; and
iv. a current controller configured to control the amount of electrical current through the conductive heating element.

24. (canceled)

25. (canceled)

26. A method of converting methane non-oxidatively, the method comprising:
(a) providing the reactor according to claim 1;
(b) contacting the NMC catalyst layer, in the reaction zone, with methane at a desired flow rate, to produce the product mixture comprising hydrogen and a C₂₊ hydrocarbon; and
c) chemically or electrically generating an amount of process heat with the source of process heat to thereby deliver the process heat to the reaction zone on the first surface, wherein the process heat creates a temperature in the range of 850° C. to about 1150° C. at the reaction zone, thereby resulting in a non-uniform temperature profile inside the reactor, where the temperature decreases at a rate in the range of 1 to 200° C./mm away from the thermal catalytic reactor member.

27.-35. (canceled)
